(19) United States
(12) Patent Application Publication
(10) Pub. No.: US 2006/0088579 A1
(43) Pub. Date: Apr. 27, 2006

(54) Transdermal drug delivery systems

(76) Inventors: Venkatram Prasad Shastri, Nashville, TN (US); Philip J. Lee, Northridge, CA (US); Samir Mitragotri, Goleta, CA (US); Robert S. Langer, Newton, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 10/503,826

(22) PCT Filed: Feb. 7, 2003
(86) PCT No.: PCT/US03/03769

§ 371 Date: Aug. 18, 2005

Related U.S. Application Data
(60) Provisional application No. 60/355,555, filed on Feb. 7, 2002.

(51) Int. Cl. A61F 13/02; A61L 15/16

(52) U.S. Cl. 424/448

(57) ABSTRACT

One aspect of the invention provides a transdermal delivery system including a drug formulated with a transport chaperone moiety that reversibly associates with the drug. The chaperone moiety is associated with the drug in the formulation so as to enhance transport of the drug across dermal tissue and releasing the drug after crossing said dermal tissue.

### 1. BACKGROUND OF THE INVENTION

Transdermal drug delivery offers a variety of advantages over oral and intravenous dosage. These include sustained release directly to the bloodstream over a long period of time, bypass of the gastrointestinal and hepatic elimination pathways, high patient compliance, and an easily administered dosage form that is portable and inexpensive.¹ Passive drug transport across human skin is governed by Fick's Law of diffusion. The mass transport equation is given as:

$$J = \frac{1}{A} ( \frac{ⅆ M}{ⅆ t} ) = P Δ C$$

where J is flux (μg cm⁻² hr⁻¹), A is cross sectional area of the skin membrane (cm²), P is the apparent permeability coefficient (cm hr⁻¹), ΔC is the concentration gradient across the membrane, and (dM/dt) is the mass transport rate. Research in the area of transdermal drug delivery has led to the commercial production of patches for nitroglycerin,² estrogen,³ testosterone,⁴ and nicotine.⁵ Although these represent major advances for transdermal delivery, these formulations rely primarily on the natural diffusion of the drug from solution into the bloodstream. Most drugs show greatly reduced diffusivity through human skin, and thus will not achieve therapeutic concentrations in the blood. Examples of such drugs include large molecular weight drugs⁶ and ionic, hydrophilic drugs.⁷
¹Walters, K. A. In Penetration enhancers and their use in transdermal therapeutic systems; Hadgraft, J.; Guy, R.; Eds.; Marcel Dekker: New York, 1989, 197-246

²U.S. Pat. No. 5,670,164

³U.S. Pat. No. 6,143,319

⁴U.S. Pat. No. 5,422,119

⁵U.S. Pat. No. 5,633,008

⁶Mitragotri, Samir; Johnson, Mark E.; Blankschtein, Daniel; Langer, Robert. Biophysical Journal. 1999, 77, 1268-1283.

⁷Gorukanti, Sudhir R.; Li, Lianli; Kim, Kwon H. Int. J. Pharm. 1999, 192, 159-172

One method to counteract this drawback is to include chemical permeation enhancers. These components are soluble in the formulation and act to reduce the barrier properties of human skin. The list of potential skin permeation enhancers is long, but can be broken down into three general categories: lipid disrupting agents (LDAs), solubility enhancers, and surfactants. LDAs are typically fatty acid-like molecules proposed to fluidize lipids in the human skin membrane.^{8,9} Solubility enhancers act by increasing the maximum concentration of drug in the formulation, thus creating a larger concentration gradient for diffusion. Surfactants are amphiphilic molecules capable of interacting with the polar and lipid groups in the skin.
⁸Francoeur, Michael L.; Golden, Guia M.; Potts, Russell O. Pharm. Res. 1990, 7, 621-627

⁹U.S. Pat. No. 5,503,843

Another field of research drawn on by this invention pertains to microemulsion (ME) systems. Characteristics of such systems are sub-micron droplet size, thermodynamic stability, optical transparency, and solubility of both hydrophilic and hydrophobic components.¹⁰ ME systems have been investigated as transdermal drug delivery vehicles, and have been found to exhibit improved solubility of hydrophobic drugs as well as sustained release profiles.
¹⁰Lawrence, M. J., et. al. Int. Journal of Pharmaceutics. 1998, 111, 63-72

Previous applications of chemical enhancers relevant to this invention include formulations containing NMP,¹¹ oleic acid,¹² lidocaine,¹³ and microemulsion based systems.¹⁴
¹¹U.S. Pat. No. 5,449,670

¹²U.S. Pat. No. 6,106,856

¹³U.S. Pat. No. 5,900,249

¹⁴U.S. Pat. No. 5,833,647

### SUMMARY OF THE INVENTION

One aspect of the invention provides a transdermal delivery system including a drug formulated with a transport chaperone moiety that reversibly associates with the drug. The chaperone moiety is associated with the drug in the formulation so as to enhance transport of the drug across dermal tissue and releasing the drug after crossing said dermal tissue.

The chaperone moiety and drug can be associated, for example, by ionic, hydrophobic, hydrogen-bonding and/or electrostatic interactions. In certain exemplary embodiments, the transport chaperone is n-methyl pyrrolidone (NMP), octadecene, isopropyl myristate (IPM), oleyl alcohol, oleic acid or a derivative thereof.

In certain exemplary embodiments, the drug is a lidocaine, a prilocaine, an estradiol or a diltiazem. In certain exemplary embodiments, the drug is a free base, such as lidocaine HCl, lidocaine free base, prilocaine HCl, estradiol or diltiazem HCl.

Another aspect of the invention provides a microemulsion system for transdermal delivery of a drug, which system solubilizes both hydrophilic and hydrophobic components. For instance, the microemulsion can be a cosolvent system including a lipophilic solvent and an organic solvent. Exemplary cosolvents are NMP and IPM.

In certain preferred embodiments, the microemulsion system has an aqueous phase, a hydrophobic organic phase, and a surfactant phase. For instance, the microemulsion system has an aqueous phase of water and ethanol, an organic phase of isopropyl mystate (IPM) and a surfactant phase of Tween 80. Another example of a microemulsion system has an aqueous phase of water and ethanol, an organic phase of IPM, and a surfactant phase is Tween 80 and Span 20.

In certain preferred embodiments, the microemulsion system is a water-in-oil system.

### BRIEF DESCRIPTION OF THE DRAWINGS

FIG. 1: Transport Chaperone Hypothesis

FIG. 2: Schematic of Drug Delivery from Multi-Phasic System

FIG. 3: NMP Chaperoning of Lidocaine Free Base from an Organic (IPM) Solvent

FIG. 4: NMP Chaperone of Lidocaine Free Base from an Aqueous (H₂O) Solvent

FIG. 5: ME System 1

FIG. 6: System 2

FIG. 7: System 3

FIG. 8: System 4

FIG. 9: Ethanol and NMP as Partitioning Agents

FIG. 10: IPM/NMP System for the Delivery of Lidocaine Free Base

FIG. 11: H₂O/NMP System for Lidocaine Free Base Delivery

FIG. 12: O/W ME Transport of Lidocaine Free Base Across Stripped Human Cadaver Skin

FIG. 13: O/W ME Transport of Lidocaine HCl Across Stripped Human Cadaver Skin

FIG. 14: O/W ME Transport of Estradiol Across Stripped Human Cadaver Skin

FIG. 15: O/W ME Transport of Diltiazem HCl Across Stripped Human Cadaver Skin

FIG. 16: IPM/NMP Binary Vehicles Through Stripped Human Cadaver Skin

FIG. 17: Correlation of NMP and Lidocaine Steady State Flux Across Stripped Human SC

FIG. 18: Correlation of NMP and Lidocaine Fluxₛₛ in NMP Solvent with LDAs

FIG. 19: Lidocaine Free Base Flux Through Stripped Human Cadaver Skin in H₂O/NMP Cosolvent

FIG. 20: Phase Diagram of Water:Ethanol:IPM:Tween 80 Microemulsion

FIG. 21: Phase Diagram of Water:Ethanol:IPM:Tween 80:Span 20 Microemulsion System

FIG. 22: Phase Diagram of Water:IPM:Tween 80:Ethanol Microemulsion System

FIG. 23: Phase Diagram of Water:IPM:Tween 80:Ethanol Microemulsion System

FIG. 24: Estradiol Transport Across Stripped Human Skin in ME Formulation

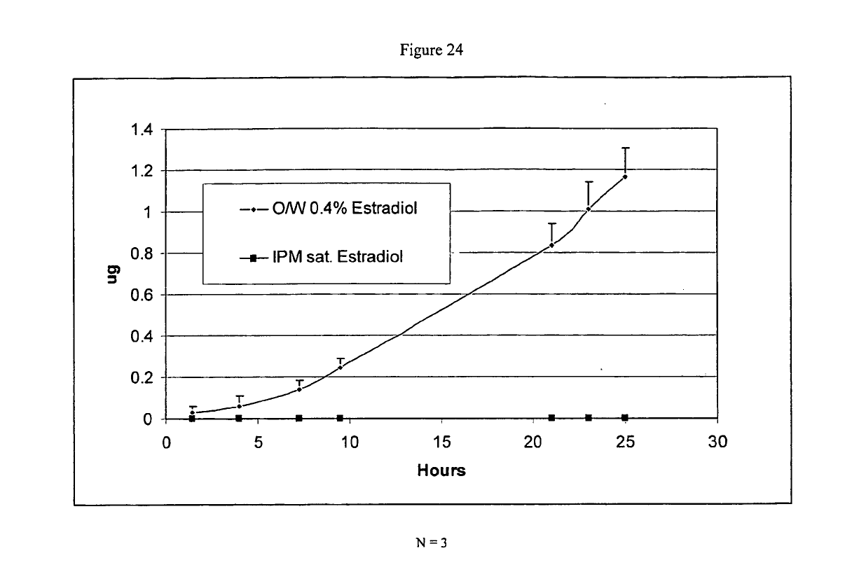

FIG. 25: Diltiazem HCl Transport Across Stripped Human Skin in ME Formulation

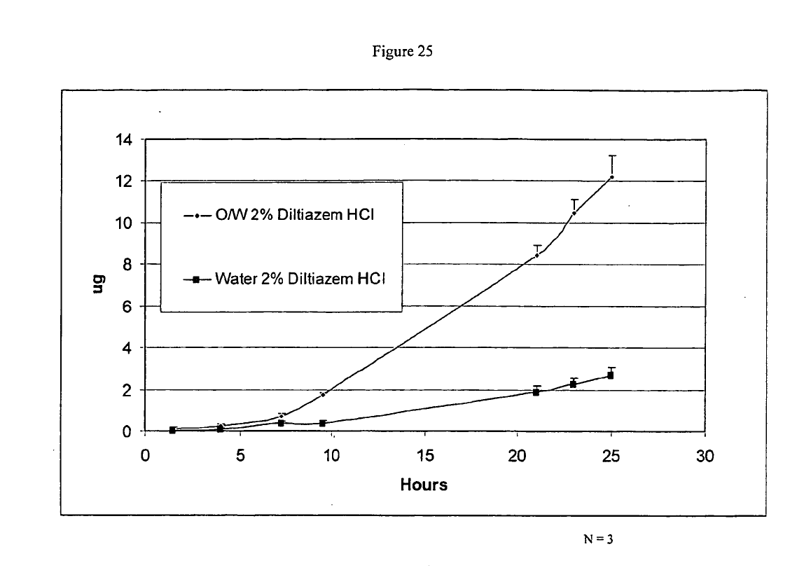

FIG. 26: Effect of NMP on Lidocaine Partitioning

### DETAILED DESCRIPTION OF THE INVENTION

The novelty of our invention is the systematic incorporation of permeation enhancers to create robust drug delivery vehicles. There are three key advances set forth in this invention. 1) A hypothesis driven schematic by which transdermal drug delivery can be enhanced. 2) The incorporation of proven permeability enhancers which meet the specifications of the hypothesis, thus providing specific enhancement systems based on the proposed schematic. 3) The enhanced delivery of select drugs utilizing the novel systems.

### EXAMPLE 1

### Chaperone-Mediated Transport for Transdermal Delivery

A. Mechanism to Enhance Transdermal Drug Delivery

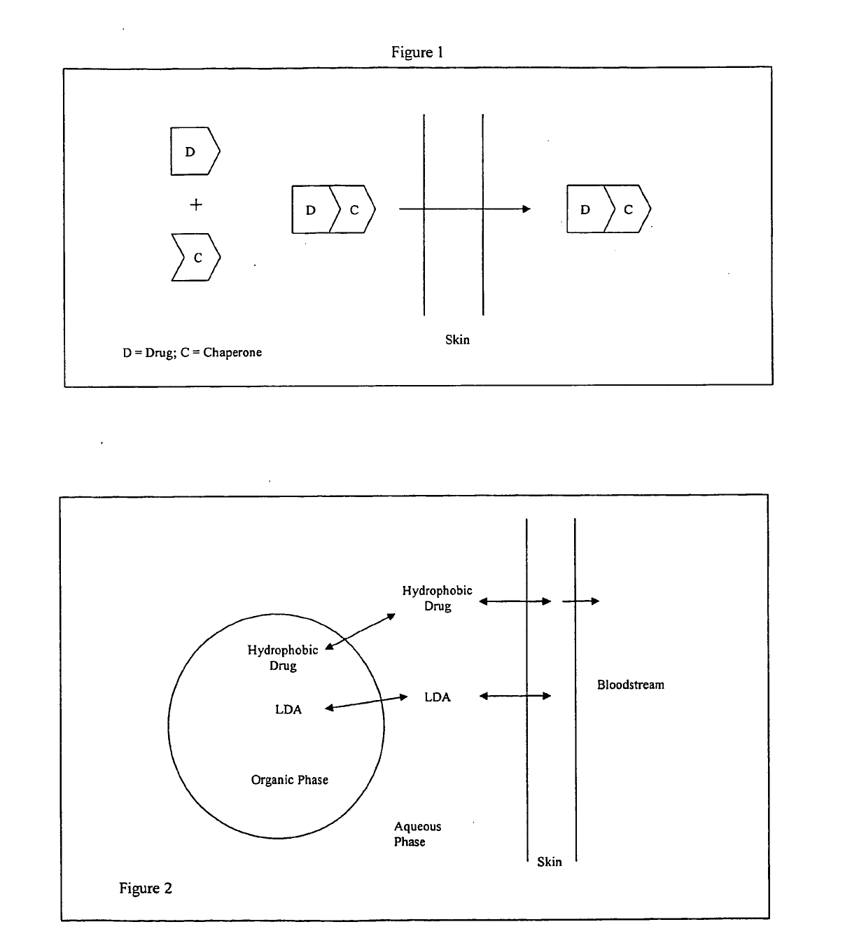

The basis of our hypothesis for enhancement is the idea of a transport chaperone (FIG. 1). An ideal chaperone molecule should have the following properties: high affinity to the drug, solubility in multiple vehicles, rapid permeation through the skin. The chaperone will reversibly bind to the drug molecule in the formulation. Because of the inherent permeation of the chaperone through the skin, it will be able to “pull” the drug across the skin into the bloodstream. As the complex is diluted in the bloodstream, the interaction will reverse and the drug will be released. In this model, a drug was chaperoned into and across the skin. The same effect could also occur between the chaperone and another permeation enhancer (such as LDA) to improve its effect.

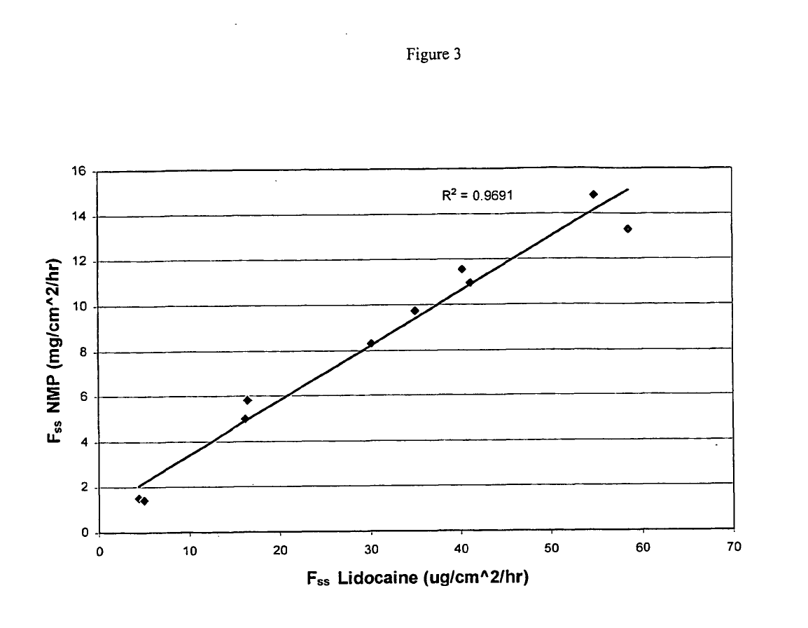

Our hypothesis further extends to a biphasic formulation, namely an O/W microemulsion. The advantage of having a biphasic system is the ability to solubilize both hydrophilic and hydrophobic components. In this system, the hydrophobic drug must first leave the organic phase and into the bulk aqueous phase (FIG. 2). This is accomplished through a partitioning agent which increases the concentration of drug in the outer, aqueous phase. Once in the aqueous phase, the chaperone described above can enhance transport through the skin. Notice that an aqueous phase chaperone is also capable of enhancing LDA activity and hydrophilic drugs from the O/W ME.

B. Proof of Principle

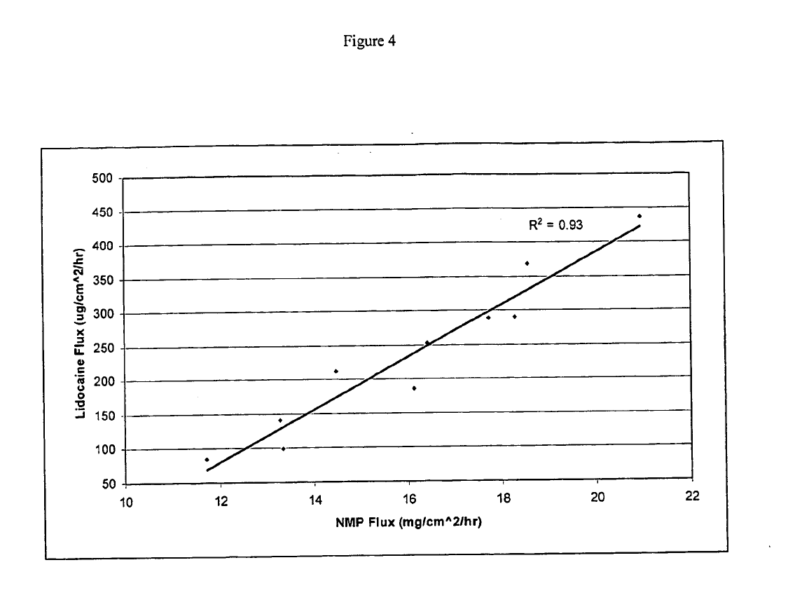

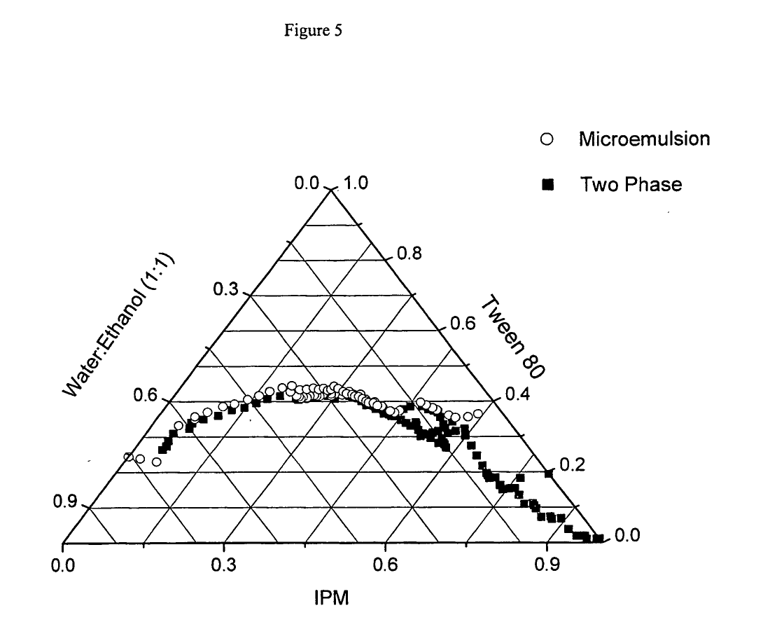

Our hypothesis was verified through in vitro drug flux studies across stripped human cadaver skin. For the permeation chaperone, n-methyl pyrrolidone (NMP) was selected as the model molecule due to its miscibility with both organic and aqueous phases, its high drug solubility, rapid flux through human skin (˜10 mg/cm²/hr), and hydrogen bonding capability (with free amine and hydroxyl groups). IR spectroscopy suggests that NMP forms hydrogen bonds with the amino group of lidocaine free base. The correlation coefficient between lidocaine free base flux and NMP flux across human skin was large from both an organic (isopropyl myristate, IPM) solvent (r²=0.97, FIG. 3) as well as from a H₂O solvent (FIG. 4, r²=0.93). This supports the claim that NMP is capable of acting as a transport chaperone for lidocaine free base. Further, the chaperoning of LDAs was tested in vitro. The enhancing effects of the molecules octadecene, IPM, oleyl alcohol, and oleic acid were determined. From an IPM bulk phase, none of these LDA-like molecules had any permeation enhancement. However, when NMP was used as the bulk solvent, there was a clear enhancement of lidocaine flux (Table 1). NMP is necessary for the LDAs to have an enhancing effect on lidocaine flux. Furthermore, the molecules capable of hydrogen bonding with NMP (oleyl alcohol and oleic acid have free hydroxyl groups) show significantly greater effect. This supports the claim that NMP aids LDA activity through the chaperone hypothesis.

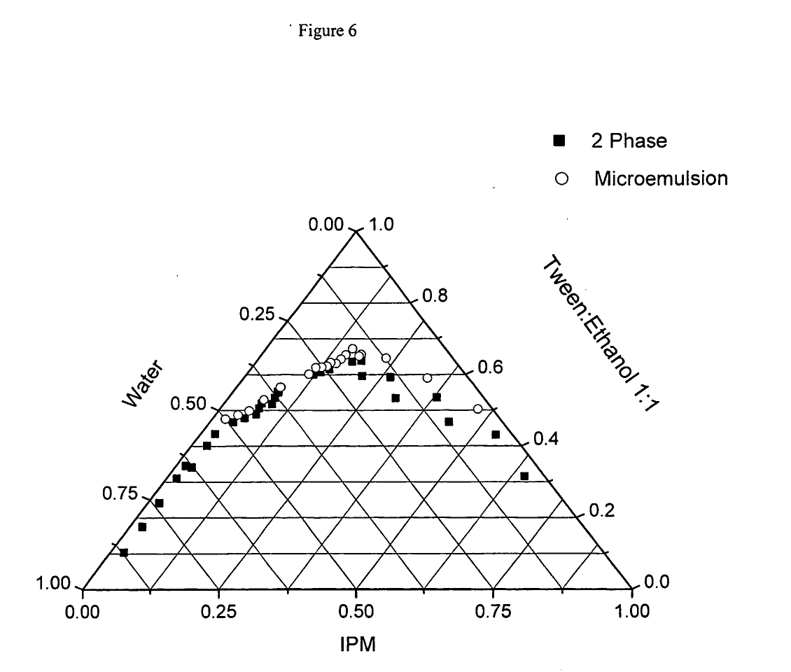

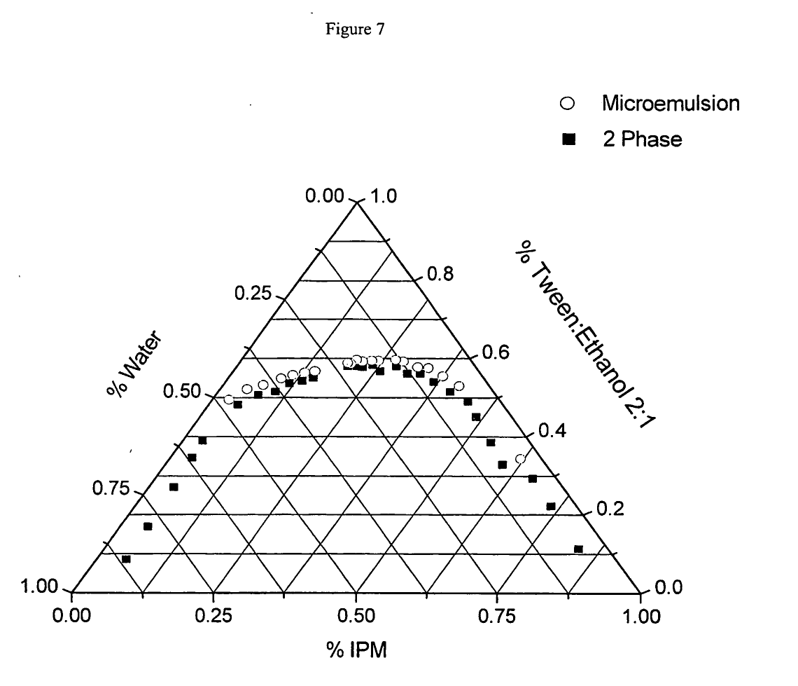

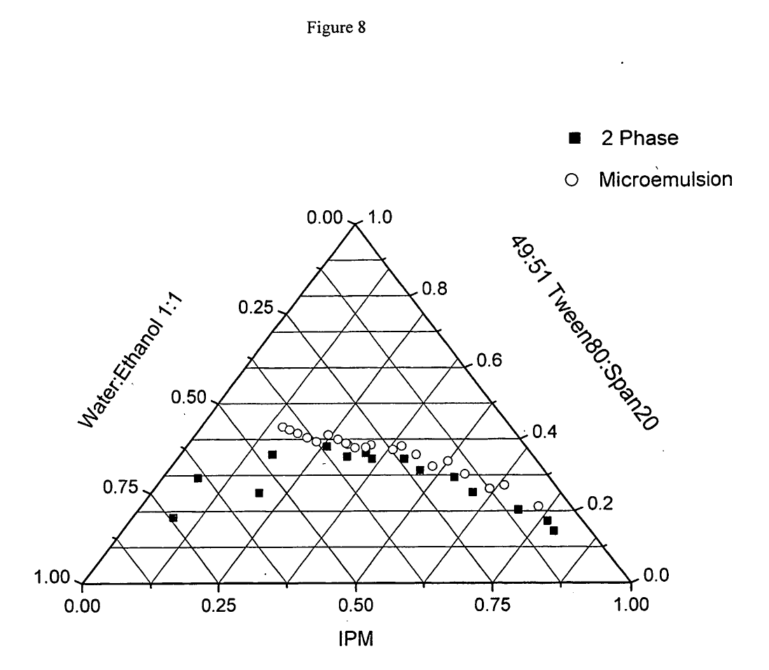

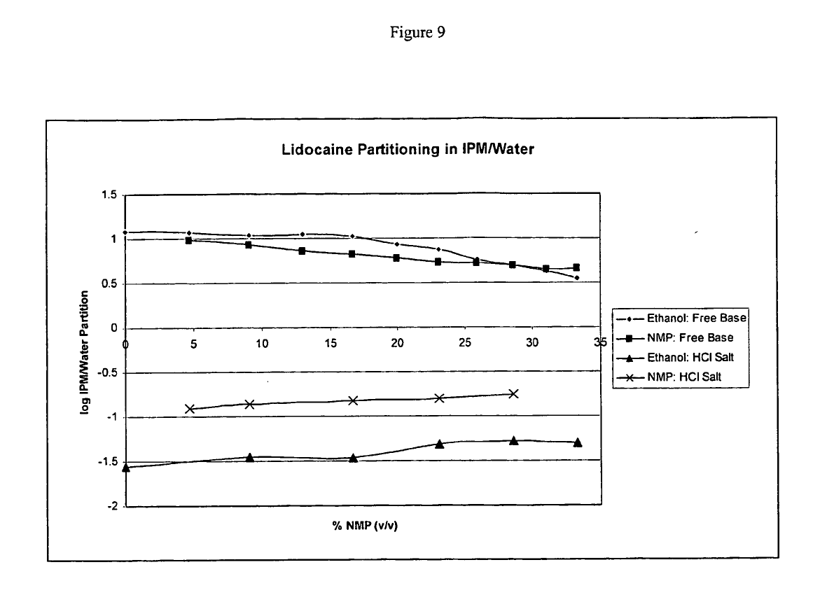

The multi-phase transport of our hypothesis was experimentally evaluated. We first described novel ME systems with the following components (FIGS. 5-8).

| | | Organic | |
|---|---|---|---|
| System | Aqueous Phase | Phase | Surfactant Phase |
| 1 | H₂O:Ethanol (1:1) | IPM | Tween 80 |
| 2 | H₂O | IPM | Tween 80:Ethanol (1:1) |
| 3 | H₂O | IPM | Tween 80:Ethanol (2:1) |
| 4 | H₂O:Ethanol (1:1) | IPM | Tween 80:Span 20 (49:51) |

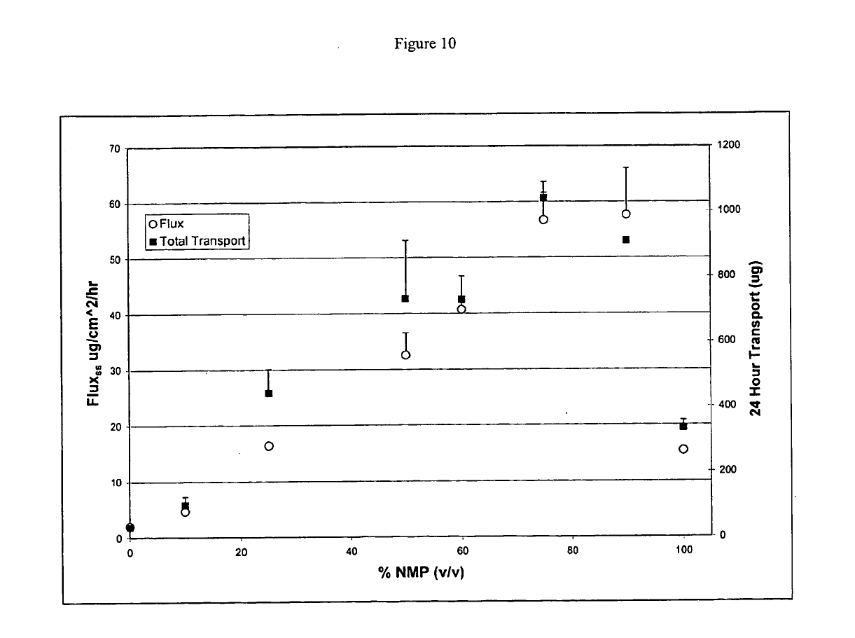

All ME systems were able to dissolve 10% w/w of NMP, oleyl alcohol, as well as other enhancers, and a maximum load of ˜30% lidocaine free base and ˜25% lidocaine HCl. Both NMP and ethanol were viable as partition agents described above. These solvents were able to increase the concentration of hydrophobic drugs in the aqueous phase and that of hydrophilic drugs in the organic phase (FIG. 9). Further support of this hypothesis was observed in the finding that O/W microemulsions exhibited greater flux than W/O microemulsion, owing to the hypothesis that NMP works primarily from the water phase. This is supported by the finding that the [IPM]/[H₂O] partition coefficient of NMP is 0.02. Additionally, flux from just the water phase of the system (with the surfactants and organic phase removed) is statistically equivalent to the O/W ME flux (Table 2). This indicates that the water phase is the dominant mode of enhancement by NMP. Another interesting feature of the O/W system is that the simultaneous delivery of both hydrophilic (diltiazem HCl) and hydrophobic (estradiol) drugs is not diminished from either drug alone (Table 2).
C. Drug Transport Profiles

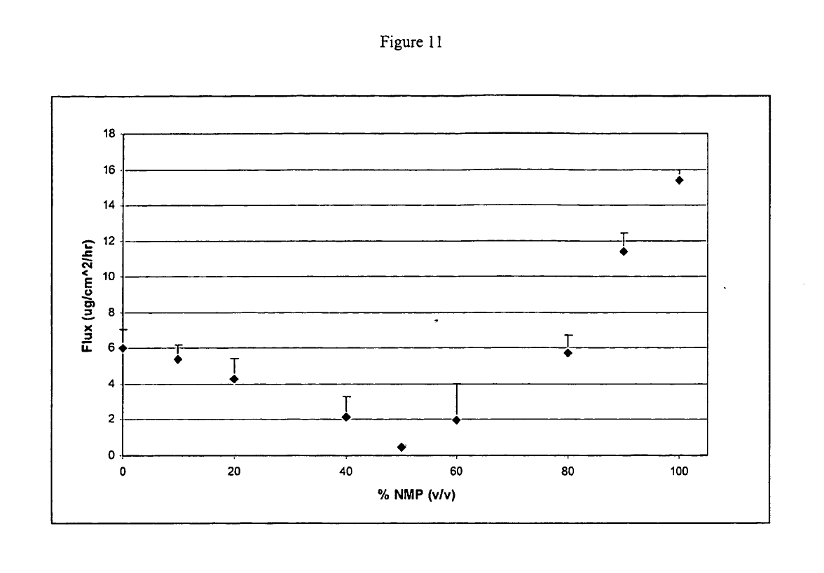

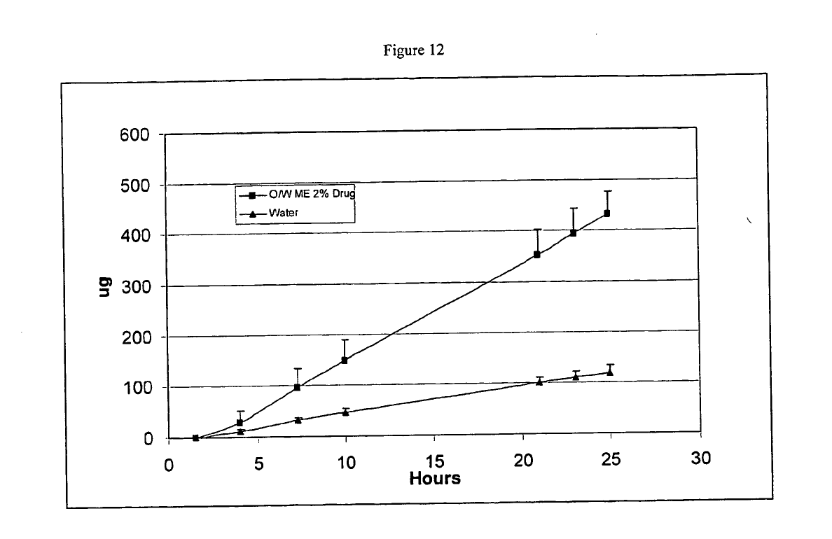

The systems described above were applied to the delivery of a number of drugs. Formulations utilizing the NMP transport chaperone principle were created in IPM and H₂O. Both the IPM/NMP system (FIG. 10) and the H₂O/NMP system (FIG. 11) showed improved drug delivery characteristics for the model hydrophobic drug lidocaine free base. The H₂O/NMP system was also found to be capable of providing enhancement for hydrophilic ionic salt drugs (Table 3).

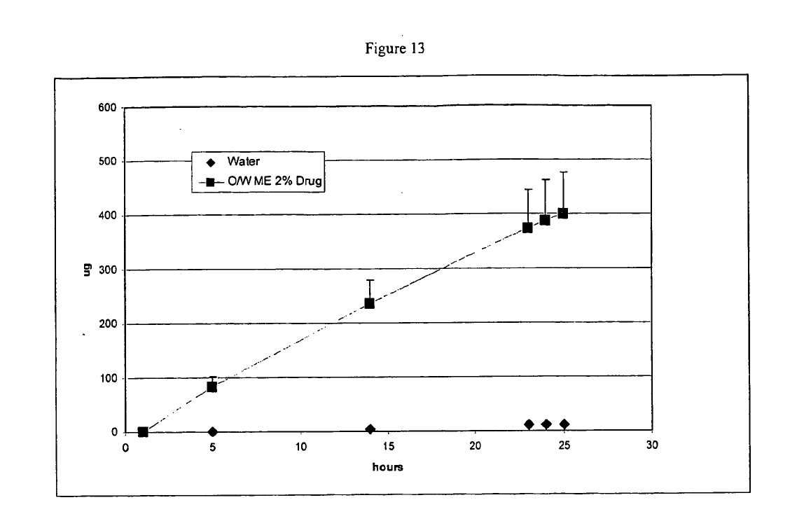

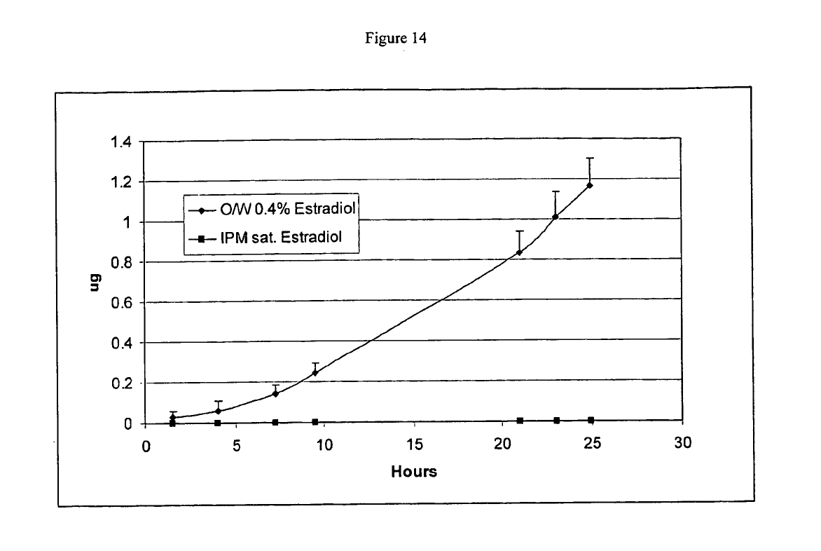

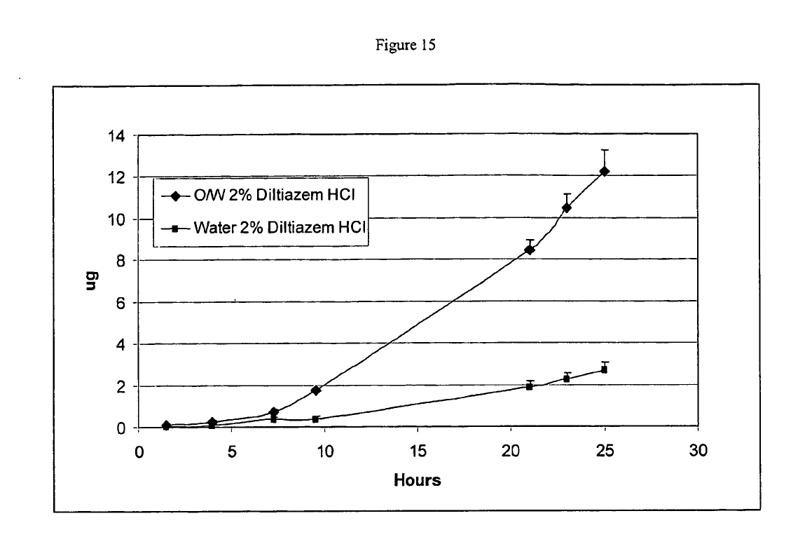

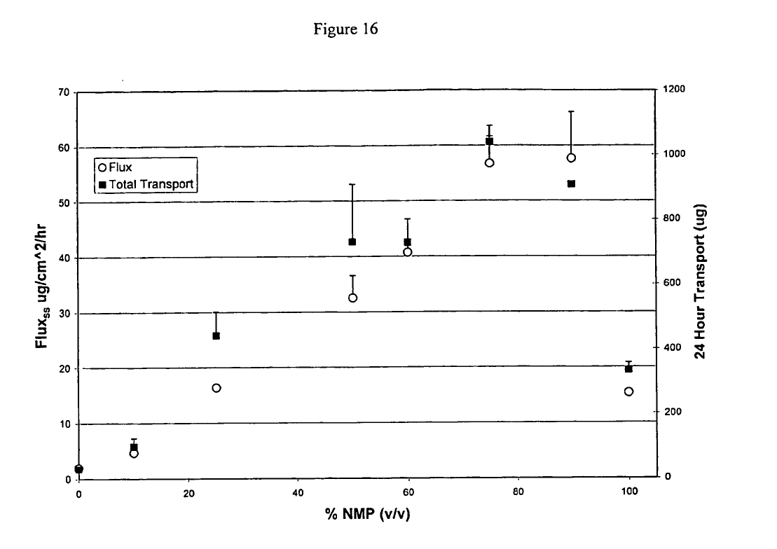

Both the W/O and O/W ME systems were evaluated for drug delivery. This systems was able to provide significant permeability enhancement for all drugs tested (Table 4). Transport profiles for lidocaine free base (FIG. 12), lidocaine HCl (FIG. 13), estradiol (FIG. 14), and diltiazem HCl (FIG. 15) indicate that steady state is reached in vitro at about 4 hours.

D. Tables

TABLE 1
NMP Synergy with LDAs

| | IPM Solvent | NMP Solvent | |
|---|---|---|---|
| | Lidocaine Fluxₛₛ | Lidocaine Fluxₛₛ | NMP Fluxₛₛ |
| LDA (1% w/v) | (μg/cm²/hr ± SD) | (μg/cm²/hr ± SD) | (mg/cm²/hr ± SD) |
| None | 1.95 ± 0.22 | 92 ± 15 | 12.6 ± 0.5 |
| Octadecene | 1.98 ± 0.58 | 161 ± 52 | 15.3 ± 1.2 |
| Isopropyl | — | 232 ± 120 | 15.8 ± 3.6 |
| Myristate | | | |
| Oleic Acid | 1.68 ± 0.24 | 290 ± 103 | 17.6 ± 2.0 |
| Oleyl Alcohol | 1.97 ± 0.48 | 402 ± 52 | 20.7 ± 1.0 |

TABLE 2
Estradiol and Diltiazem HCl Transport from ME Systems

| | Estradiol | | Diltiazem HCl | |
|---|---|---|---|---|
| Formu- | Fluxₛₛ | Permeability | Fluxₛₛ | Permeability |
| lation | (μg/cm²/hr) | (cm/hr · 10⁵) | (μg/cm²/hr) | (cm/hr · 10⁵) |
| H₂O | 0.015 ± 0.006 | 460 ± 183 | 0.05 ± 0.01 | 0.015 ± 0.004 |
| W/O ME | 0.053 ± 0.029 | 1.1 ± 0.6 | 0.25 ± 0.13 | 1.2 ± 0.6 |
| W/O ME | 0.12 ± 0.06 | 2.4 ± 1.2 | 0.24 ± 0.08 | 1.2 ± 0.4 |
| Both | | | | |
| Drugs | | | | |
| O/W ME | 0.27 ± 0.07 | 5.8 ± 1.5 | 1.6 ± 0.3 | 7.8 ± 1.3 |
| O/W ME | 0.23 ± 0.05 | 5.0 ± 1.2 | 1.6 ± 0.4 | 7.8 ± 1.9 |
| Both | | | | |
| Drugs | | | | |
| Water | | 6.5 ± 1.7 | | 6.1 ± 3.7 |
| Phase | | | | |

TABLE 3
Flux Enhancement of Hydrophilic HCl Salt Drugs from 1:1
H2O/NMP Cosolvent through Stripped Human Cadaver Skin

| | | Fluxₛₛ from | |
|---|---|---|---|
| | Fluxₛₛ from H₂O | H₂O/NMP | |
| Drug (2% w/v) | (μg/cm²/hr ± SD) | (μg/cm²/hr ± SD) | Enhancement |
| Lidocaine HCl | 1.00 ± 0.22 | 4.35 ± 1.84 | 4.3 ± 2.1 |
| Prilocaine HCl | 2.44 ± 0.98 | 6.31 ± 0.14 | 2.6 ± 1.0 |

TABLE 4
Permeability Enhancement of ME Systems

| | Permeability | |
|---|---|---|
| | (cm/hr · 10⁵) | Enhancement |
| Estradiol | | |
| IPM | <0.1 | |
| W/O ME | 1.1 ± 0.6 | >11 |
| O/W ME | 5.8 ± 1.5 | >58 |
| Diltiazem HCl | | |
| H₂O | 0.015 ± 0.004 | |
| W/O ME | 1.2 ± 0.6 | 80 |
| O/W ME | 7.8 ± 1.3 | 520 |
| Lidocaine Free Base | | |
| IPM | 7.2 ± 1.1 | |
| W/O ME | 40.1 ± 4.5 | 5.6 |
| O/W ME | 123 ± 36 | 17 |
| Lidocaine HCl | | |
| H₂O | 0.61 ± 0.38 | |
| W/O ME | 3.5 ± 0.3 | 5.7 |
| O/W ME | 18.1 ± 6.9 | 30 |

E. References for Example 1
1. Walters, K. A. In Penetration enhancers and their use in transdermal therapeutic systems; Hadgraft, J.; Guy, R.; Eds.; Marcel Dekker: New York, 1989, 197-246
2. U.S. Pat. No. 5,670,164
3. U.S. Pat. No. 6,143,319
4. U.S. Pat. No. 5,422,119
5. U.S. Pat. No. 5,633,008
6. Mitragotri, Samir; Johnson, Mark E.; Blankschtein, Daniel; Langer, Robert. Biophysical Journal. 1999, 77, 1268-1283.
7. Gorukanti, Sudhir R.; Li, Lianli; Kim, Kwon H. Int. J. Pharm. 1999, 192, 159-172
8. Francoeur, Michael L.; Golden, Guia M.; Potts, Russell O. Pharm. Res. 1990, 7, 621-627
9. U.S. Pat. No. 5,503,843
10. Lawrence, M. J., et. al. Int. Journal of Pharmaceutics. 1998, 111, 63-72
11. U.S. Pat. No. 5,449,670
12. U.S. Pat. No. 6,106,856
13. U.S. Pat. No. 5,900,249
14. U.S. Pat. No. 5,833,647

### EXAMPLE 2

### Evaluation of Chemical Enhancers in the Transdermal Delivery of Lidocaine

Lidocaine free base is a local anesthetic routinely used in topical applications. This study aims at investigating the effect of various classes of chemical enhancers on in vitro drug transport across human and pig skin. The lipid disrupting agents oleic acid, oleyl alcohol, butene diol, and decanoic acid show no significant flux enhancement. The binary system of isopropyl myristate/n-methyl pyrrolidone (IPM/NMP) exhibits a marked synergistic effect on drug transport. This effect peaks at 25:75 v/v IPM:NMP reaching a steady state flux of 57.6±8.4 μg cm⁻² hr⁻¹ through human skin. This is 4-fold enhancement over a NMP solution and over 25-fold increase over IPM (p<0.001). There is a tight correlation of lidocaine flux with NMP flux (r²=0.97) over the range of NMP≦75%. IR spectroscopy analysis of lidocaine solutions indicates that it forms hydrogen bonds in the presence of NMP solvent. This suggests that NMP may act as a “transport chaperone,” capable of enhancing the flux of drug molecules if delivered in the IPM/NMP system.

A. Introduction.

Lidocaine is a widely used local anesthetic for a variety of medical procedures including treatment of open skin sores and lesions, surgical procedures such as suturing of wounds, and venipuncture.¹⁵ Lidocaine is also a first line anti-arrhythmic drug when administered to the heart in larger doses.¹⁶ The most common method of lidocaine delivery is through IV or hypodermic injection. When lidocaine is injected as an analgesic agent, the discomfort caused by the application is counterproductive to the pain relieving effect of the drug. For purposes such as preparation for pediatric venipuncture, a painless means to administer lidocaine to the site of injection would be an important procedure. This makes local transdermal delivery of lidocaine a likely avenue of research. Transdermal lidocaine products such as EMLA® cream (AstraZeneca) and Lidoderm® (Endo Laboratories) are commercially available. However, further improvement in enhancement of transdermal lidocaine delivery is still desired.
¹⁵Smith, D W; Peterson, M R; DeBerard, S C. Postgraduate Medicine. 1999, 106(2), 57-60, 64-66

¹⁶Sleight, P. J. Cardiovasc. Pharmacol. 1990, 16, Suppl 5: S113-119

The primary barrier to transdermal drug delivery is the outermost layer of the skin, the stratum corneum (SC).¹⁷ The SC consists of keratinocytes embedded in a continuous lipid phase, forming a tortuous network preventing the infiltration of exogenous agents into the body.¹⁸ A variety of methods for increasing transdermal drug transport are currently studied. These include chemical enhancers,¹⁹ therapeutic and low frequency ultrasound,²⁰ iontophoresis,²¹ and electroporation.²² While all these methods are capable of providing significant enhancement of drug delivery, a simple passive system free of additional machinery would prove most effective for the local delivery of lidocaine. In this study, the effects of a variety of chemical permeation enhancers are evaluated for the transdermal delivery of lidocaine free base.
¹⁷Ranade, Vasant V. J. Clin. Pharmacol. 1991, 31, 401-418

¹⁸Johnson, Mark E.; Blankschtein, Daniel; Langer, Robert. Journal of Pharmaceutical Sciences. 1997, 86, 1162-1172

¹⁹Walters, K. A. In Penetration enhancers and their use in transdermal therapeutic systems; Hadgraft, J.; Guy, R.; Eds.; Marcel Dekker: New York, 1989, 197-246

²⁰Mitragotri, S.; Blankschtein, D.; Langer, R. Science. 1995, 269, 850-853

²¹Burnette, R. R. In Iontophoresis; Hadgraft, J.; Guy, R. H.; Eds.; Marcel Dekker: New York, 1989; 247-291

²²Prausnitz, M. R.; Bose, V. G.; Langer, R.; Weaver, J. C. PNAS 1993, 90, 10504-10508

Chemical enhancers with different proposed mechanisms of action were tested for their effects alone and in combination. Two main transport pathways have been proposed through human SC—the polar, aqueous pathway and the lipid pathway.¹⁷ The majority of research on transdermal drug transport to date has been focused on delivery through the continuous lipid region of the SC. Furthermore, transport of drug through the aqueous pathway has proven very difficult.²³ Since lidocaine free base is a lipophilic molecule (log octanol-water partition coefficient=2.48), it is reasonable to explore lipid pathway enhancing chemicals to improve drug flux. The more commonly studied chemical enhancers can be broken down into 3 broad categories. The first is the class of lipid disrupting agents (LDAs), usually consisting of a long hydrocarbon chain with a cis-unsaturated carbon-carbon double bond.^{24,25} These molecules have been shown to increase the fluidity of the SC lipids, thereby increasing drug transport. In this study, oleic acid, oleyl alcohol, decanoic acid, and butene diol were investigated as lipid disrupting agents. A second class of permeation enhancers relies on improving drug solubility and partitioning into the skin.²⁶ The lipophilic vehicle isopropyl myristate (IPM)²⁷ as well as the organic solvents ethanol²⁸ and N-methyl pyrrolidone (NMP)²⁹ were studied. A final class of enhancers consists of surfactants. These molecules have affinity to both hydrophilic and hydrophobic groups, which might facilitate in traversing the complex regions of the SC. An anionic surfactant lauryl sulfate (SDS) and a nonionic surfactant polysorbate 80³⁰ (Tween 80) was tested for their effect on lidocaine delivery.
¹⁷Ranade, Vasant V. J. Clin. Pharmacol. 1991, 31, 401-418

²³Peck, Kendall D.; Ghanem, Abdel-Halem; Higuchi, William I. J. Pharm. Sci. 1995, 84, 975-982

²⁴Francoeur, Michael L.; Golden, Guia M.; Potts, Russell O. Pharm. Res. 1990, 7, 621-627

²⁵Kim, Dae-Duk; Chien, Yie W. J. Pharm. Sci. 1996, 85, 214-219

²⁶Guy, Richard H.; Hadgraft, Jonathan. J. Controlled Release. 1987, 5, 43-51

²⁷Gorukanti, Sudhir R.; Li, Lianli; Kim, Kwon H. Int. J. Pharm. 1999, 192, 159-172

²⁸Liu, Puchun; Kurihara-Bergstrom, Tamie; Good, William R. Pharm. Res. 1991, 8, 938-944

²⁹Yoneto, Kunio; Li, S. Kevin; Ghanem, Abdel-Halim; Crommelin, Daan J. A.; Higuchi, William I. J. Pharm. Sci. 1995, 84, 853-860

³⁰Sarpotdar, Pramod P.; Zatz, Joel L. J. Pharm. Sci. 1986, 75, 176-181

It has been reported in the literature that combinations of various enhancers result in a synergistic increase in drug flux that is far greater than either chemical by itself.^{31,32} Various combinations of enhancer combinations were tested to identify useful trends in lidocaine free base delivery. Because of the different solubility of lidocaine free base in each of the delivery vehicles, saturated solutions were utilized in each sample to maintain a constant thermodynamic activity of the drug.
³¹Johnson, Mark E.; Mitragotri, Samir; Patel, Ashish; Blankschtein, Daniel; Langer, Robert. J. Pharm. Sci. 1996, 85, 670-679

³²Sasaki, Hitoshi; Kojima, Masaki; Nakamura, Junzo; Shibasaki, Juichiro. J. Pharm. Pharmacol. 1990, 42, 196-199

Lidocaine free base is a commonly studied drug for transdermal delivery^{17,33} as its hydrophobicity and molecular size (MW 234.3) characterize it as a typical transdermal drug candidate. By studying the effects of a wide range of chemical enhancers across both full thickness pig skin and stripped human cadaver skin, some general trends of transdermal permeation enhancement can be hypothesized.
¹⁷Ranade, Vasant V. J. Clin. Pharmacol. 1991, 31, 401-418

³³Johnson, Mark E., Blankschtein, Daniel; Langer, Robert. J. Pharm. Sci. 1995, 84, 1144-1146

B. Materials

Drug: Lidocaine free base was purchased from Sigma (St. Louis, Mo.). Chemicals: NMP was a generous gift from ISP Technologies, Inc. (Wayne, N.J.). USP grade oleic acid was purchased from Mednique. Polysorbate 80 NF (Tween 80) was purchased from Advance Scientific & Chem. (Ft. Lauderdale, Fla.). Isopropyl myristate (IPM), oleyl alcohol (99%), anhydrous ethyl alcohol, SDS, cis-2-butene-1,4-diol, decanoic (capric) acid, and phosphate buffered saline tablets (PBS) were purchased from Sigma (St. Louis, Mo.). HPLC grade solvents were used as received. Skin: Human cadaver skin from the chest, back, and abdominal regions was obtained from the National Disease Research Institute (Philadelphia, Pa.). The skin was stored at −80° C. until use.

C. Methods

(i) Preparation of Lidocaine Solutions. Sample solutions were prepared in 20 ml glass vials and saturated with drug. In binary systems the sample contained 50% (w/w) of each liquid. All vehicles studied formed miscible, single phase liquids.

(ii) Determination of Saturation Concentration. All samples were mixed with a magnetic stir-bar in the presence of lidocaine free base crystals for at least 24 hours at room temperature. The saturated solutions were then syringe filtered through a 0.2 μm filter to remove undissolved drug. Concentrations of the filtered solutions were determined by HPLC after dilution to a suitable range.

(iii) Preparation of Skin Samples. Human cadaver skin was thawed at room temperature. The epidermis-SC was separated from the full thickness tissue after immersion in 60° C. water for 2 minutes. Heat stripped skin was immediately mounted on diffusion cells. Full thickness pig skin was prepared by removing the dermal tissue from a freshly sacrificed pig. Pig skin samples were subsequently frozen and stored at −20° C. or −80° C.

(iv) Lidocaine Transport Experiments. The skin was mounted onto a side-by-side glass diffusion cell with an inner diameter of 5 mm. The two halves of the cell were clamped shut and both reservoirs were filled with 2 ml of phosphate buffered saline (PBS, 0.01 M phosphate, 0.137 M NaCl, pH 7.4). The integrity of the skin was verified by measuring the electrical conductance across the skin barrier at 1 kHz and 10 Hz at 143.0 mV (HP 33120A Waveform Generator). Skin samples measuring 4-14 μA at 1 kHz were used for the diffusion studies. Prior to introducing the donor solution, the skin sample was thoroughly rinsed with PBS to remove surface contaminants. At t=0, the receiver compartment was filled with 2.0 ml of PBS, while 2.0 ml of sample was added to the donor compartment. Both compartments were continuously stirred to maintain even concentrations. At regular time intervals, 1.0 ml of the receiver compartment was transferred to a glass HPLC vial. The remaining solution in the receiver compartment was thoroughly aspirated and discarded. Fresh PBS (2.0 ml) was dispensed into the receiver compartment to maintain sink conditions. At 21 hours, the experiment was terminated. After both compartments were refilled with PBS, the conductance across the skin membrane was again checked to ensure that the skin was not damaged during the experiment. All flux experiments were conducted in triplicate at room temperature. The observed variability of the individual drug transport values was consistent with the previously established 40% intersubject variability of human skin.³⁴
³⁴Williams, A. C.; Cornwell, P. A.; Barry, B. W. Int. J. Pharm. 1992, 86, 69-77

(v) IPM/NMP Binary Vehicle Transport. The two miscible liquids were mixed in the specified v/v ratios, with 2% w/v lidocaine free base added. Flux cells were set up as described above. At t=4, 21, 23, 25 hours, the transport of drug across the skin was measured by HPLC. Steady state conditions were taken as the average of the final 2 time points.

(vi) Quantification of Lidocaine. Lidocaine was assayed by high pressure liquid chromatography (Shimadzu model HPLC, SCL-10A Controller, LC-10AD pumps, SPD-M10A Diode Array Detector, SIL-10AP Injector, Class VP v.5.032 Integration Software) on a reverse phase column (Waters μBondapak™ C₁₈ 3.9×150 mm) using ddH₂O (5% acetic acid, pH 4.2)/acetonitrile (35:65 v/v) as the mobile phase, under isocratic conditions (1.6 mL/min) by detection at 237 nm. The retention time of lidocaine under these conditions was between 3.4 and 4.3 minutes. Standard solutions were used to generate calibration curves. NMP was quantified on a Waters Symmetry® C₁₈ 5 μm, 3.9×150 mm column (WAT046980). The mobile phase consisted of ddH₂O:methanol (95:5) at a flow rate of 1.2 ml/min. Chromatograms were integrated at a peak of 205 nm, with retention time at 3.8-4.8 min.

(vii) Calculations. The total mass of drug transported across the skin was determined by HPLC. The flux equation gives:

$$F = \frac{1}{A} ( \frac{ⅆ M}{ⅆ t} ) = P Δ C$$

where F is flux (μg cm⁻² hr⁻¹), A is cross sectional area of the skin membrane (cm²), P is the apparent permeability coefficient (cm hr⁻¹), and ΔC is the concentration gradient. In this experiment, ΔC is taken as the saturation concentration (given infinite dose and sink conditions), and dM/dt is averaged as the total mass transport over the time course of the experiment. Statistical analyses were performed by the Student's t-test.

D. Results

(i) Lidocaine Free Base Solubility. The maximum concentration of drug in an application vehicle is an important element determining transdermal flux. According to Fick's Law of diffusion, flux across a membrane scales linearly with concentration. The maximum level of drug transport across the skin should occur when the donor solution is saturated with drug. The solubility of a drug in various solutions also gives indications of molecular interactions such as hydropobicity, hydrogen bonding capability, and pH dependence.

Due to the lipophilicity of lidocaine free base, its solubility in aqueous media was limited. The saturation concentration of lidocaine free base in water was over 60 times less than its solubility in a hydrocarbon oil such as isopropyl myristate (IPM). Although the addition of the anionic surfactant SDS (CMC=0.2%) at 10 mg/ml improved the solubility of the hydrophobic drug in water, the saturation point remained too low to provide appreciable flux. The solubility of lidocaine free base in the hydrophobic enhancers was in the 300-400 mg/ml range (Table 5). The two solvents that significantly improved the saturation concentration of lidocaine free base were ethanol (618 mg/ml) and NMP (733 mg/ml).

(ii) Permeability of Lidocaine Free Base. The in vitro permeability of lidocaine free base across stripped human skin and full thickness pig skin gives an indication of the enhancing effect of each chemical beyond their ability to improve drug saturation concentration. The permeability of lidocaine free base in saturated neat enhancer solutions is given in Table 5. Although the apparent permeability of lidocaine from the two aqueous solutions (H₂O and 1% SDS) appear significant compared to the other samples, they should be discounted as viable delivery vehicles due to their minimal lidocaine saturation point. The physical flux of lidocaine free base across human skin in vitro from water was 10.8±1.95 μg cm⁻² hr⁻¹ as compared to 20.4±3.02 μg cm⁻² hr⁻¹ from an IPM solution. This significant difference (p<0.001) makes water a poor candidate as a transdermal vehicle. A similar trend was seen in full thickness pig skin.

(iii) LDAs. The chemical enhancers with lipid-disrupting ability (oleyl alcohol, oleic acid, butene-diol) did not show significant improvement of lidocaine permeability or flux. In fact, the flux and permeability of lidocaine free base in the presence of these enhancers was either statistically equivalent or below that of IPM solution.

(iv) Solubility/Partition Enhancers. Permeability experiments across both human and pig skin indicated that the two chemical enhancers with highest drug solubility were poor permeation enhancers. The permeability of lidocaine free base in saturated solutions of ethanol and NMP did not surpass that of IPM. By themselves, these two solvents appeared to be able to increase drug flux only by improving drug solubility. These skin penetration enhancers were unable to markedly increase lidocaine free base permeability as neat enhancer solutions.

(v) Surfactants. The two surfactant enhancers studied (SDS and polysorbate 80) did not significantly increase drug flux. Among the two aqueous donors (H₂O and 1% SDS), there was no statistical difference between the permeability of lidocaine through human or pig skin. SDS was capable of increasing the solubility of lidocaine free base, but it had no noticeable enhancing effect on skin permeability. Polysorbate 80 tended to increase the viscosity of the solution and had a severely negative effect on drug transport (data not shown).

(vi) Permeability in Cosolvent Systems. To evaluate possible synergistic interaction between the studied enhancers, 1:1 ratios of selected chemicals were mixed to form cosolvent systems (Table 6). It has been previously reported that combining transdermal chemical enhancers can greatly improve drug transport through human skin.³⁵ For practical purposes, IPM was selected as the bulk oil phase to be mixed with other enhancers. It is a molecule consisting of a long hydrocarbon chain, satisfying the hypothesis that lidocaine free base is transported best through a hydrophobic vehicle. IPM is also inexpensive, easy to work with, and exhibited the best in vitro permeability of the studied enhancers. Cosolvents of IPM were made with oleyl alcohol, oleic acid, decanoic acid, and NMP. Decanoic acid is chemically similar to oleic acid, and may act in the skin as a lipid disrupting agent. In these cosolvent systems the LDAs had no enhancing effect on lidocaine free base permeability. The trend appears similar to that of the neat solvents, suggesting that mixing these LDAs with IPM did not result in pronounced enhancement. The only IPM/cosolvent system that had a significant effect on permeability across stripped human skin was the IPM/NMP system (p<0.005). Because of the higher drug solubility of this NMP containing system, the total transdermal flux across human skin (165±27 μg cm⁻² hr⁻¹) was roughly 8-fold greater than that of just an IPM vehicle, and over 6-fold better than a saturated NMP system (p<0.001). A similar trend was seen in full thickness pig skin (Table 6).
³⁵Priborsky, Jan; Takayama, Kozo; Nagai, Tsuneji; Waitzova, Danuse; Elis, Jiri. Drug Design and Delivery. 1987, 2, 91-97

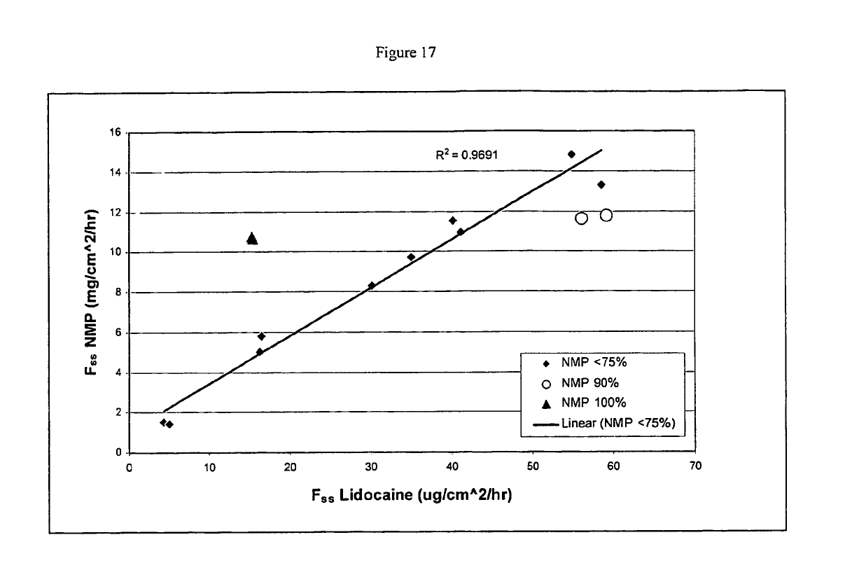

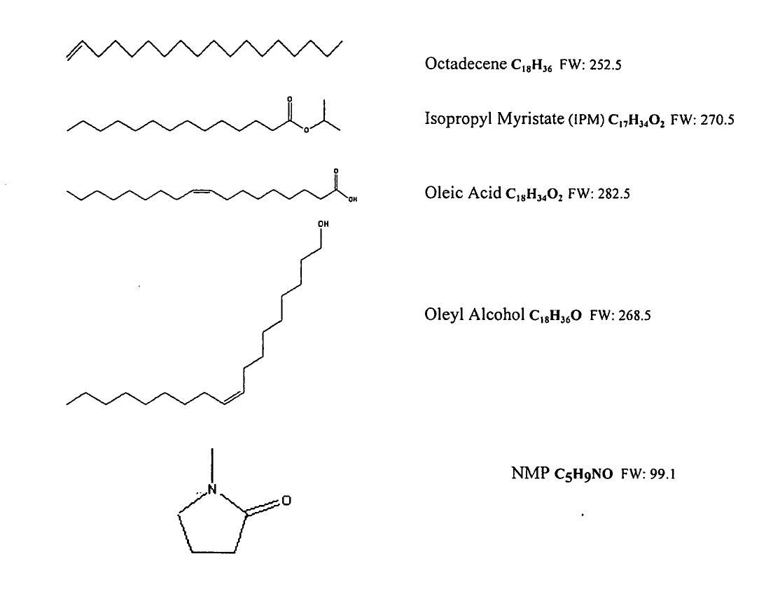

(vii) IPM/NMP Cosolvent Flux. From these experiments, an IPM/NMP cosolvent system appeared to exhibit the best flux. To further investigate this effect, the two enhancers were mixed in varying ratios in the presence of 2% lidocaine free base (Table 7). Between 10% and 75% NMP concentration, the increase in lidocaine flux scaled linearly with NMP concentration. Lidocaine flux was equivalent at 75% and 90% NMP (p>0.5), and increasing to 100% NMP reduced flux to 26% of its previous value (FIG. 16). When the flux of NMP through the skin of the same vehicles was tested, it produced similar results (FIG. 17). In the range of linear flux increase (10-75% NMP), there was a corresponding linear increase in NMP flux (r²=0.97). When NMP concentration was increased to 90% and 100%, the total NMP flux from the vehicle decreased.

(viii) IR Analysis of Lidocaine Systems. To gain a better understanding of why IPM/NMP is a good transdermal enhancer for lidocaine, IR spectra of drug solution in various vehicles was obtained. From the saturation study, it was established that NMP is the best lidocaine free base solvent of the chemicals studied. However, the permeability from the IPM/NMP cosolvent system suggests that there was an effect other than improving donor concentration. In IR spectra of lidocaine free base in NMP, the amide group band was shifted lower, suggesting hydrogen bonding by NMP. The potential of NMP to form hydrogen bonds with the amide group of lidocaine may facilitate the transport of drug through SC.

E. Discussion

This evaluation of chemical permeation enhancers on lidocaine free base transport across human skin expands the current knowledge of the effectiveness of transdermal enhancers. There currently exists a large collection of chemicals believed to enhance transdermal drug delivery, yet their benefits have been difficult to apply broadly to multiple drugs. In this study, lidocaine free base was selected as a model drug to aid in the understanding of the relative effectiveness of some of the more widely used chemical enhancers.

Due to the continuous lipid regions in the stratum corneum, it is currently believed that passive transdermal diffusion occurs predominantly through the lipid phase of the skin.^{3,4} For this reason, hydrophobic drugs generally have better transport through skin while water soluble ionic drugs have very limited permeability.³⁶ This relation holds true for lidocaine free base. Because of its limited solubility in water, the amount of drug transported from this phase is less than from an oil phase vehicle. Lidocaine also exists as a hydrophilic hydrochloride salt (lidocaine HCl). As expected, both the flux and the permeability of lidocaine HCl through human skin is an order of magnitude less than for the free base (Data not shown). Based on these observations, we decided to investigate lipophilic enhancers.
³U.S. Pat. No. 6,143,319

⁴U.S. Pat. No. 5,422,119

³⁶Lee, Cheon Koo; Uchida, Takahiro; Kitagawa, Kazuhisa; Yagi, Akira; Kim, Nak-Seo; Goto, Shigeru. J. Pharm. Sci. 1994, 83, 562-565

A common class of such enhancers is the LDAs. These hydrophobic molecules are believed to fluidize the stratum corneum lipids and reduce its barrier properties. In the case of lidocaine free base, none of these agents improved the permeability of drug across the skin. Saturated solutions of oleyl alcohol, oleic acid, butene-diol, and decanoic acid yielded very poor lidocaine free base transport. (1% oleyl alcohol in IPM also had no statistical improvement.) This result might be explained by the hypothesis that transport of relatively small molecules such as lidocaine free base are not severely hindered by lipid bilayers.^{17,37,38} Altering the bilayer properties in this case will not result in dramatic flux increases. In order to enhance lidocaine permeability, an enhancer must target a step of the transport process that is rate determining.
¹⁷Ranade, Vasant V. J. Clin. Pharmacol. 1991, 31, 401-418

³⁷Mitragotri, Samir; Johnson, Mark E.; Blankschtein, Daniel; Langer, Robert. Biophysical Journal. 1999, 77, 1268-1283.

³⁸Mitragotri, Samir. Pharm. Res. 2001, 18(7), 1018-23

NMP and its derivatives are widely used chemical enhancers which have produced significant results in the transport of various drugs.^{18,39} More recently, they have been used in conjunction with more lipophilic molecules to enhance partitioning of more hydrophilic drugs into the skin.¹⁵ Although NMP by itself is an exceptional solvent for drugs, our experiments show that it does not greatly improve the permeability of lidocaine free base. However, combining NMP with IPM results is in substantial flux improvement. In 2% lidocaine systems, the maximum flux occurs between 75% and 90% NMP, with a linear relationship below 75% NMP. This relationship may be useful by allowing the control of drug flux by adjusting NMP concentration in the vehicle.
¹⁸Johnson, Mark E.; Blankschtein, Daniel; Langer, Robert. Journal of Pharmaceutical Sciences. 1997, 86, 1162-1172

³⁹Phillips, Christine A.; Michniak, Bozena B. J. Pharm. Sci. 1995, 84, 1427-1433

¹⁵Smith, D W; Peterson, M R; DeBerard, S C. Postgraduate Medicine. 1999, 106(2), 57-60, 64-66

The synergy observed in saturated IPM/NMP systems in vitro could be explained by an osmotic pressure gradient into the donor compartment that retards the flux of drug. NMP is freely miscible in water, and has a high affinity for it. An osmotic gradient is created between the donor compartment (>700 mg/ml lidocaine free base) and the receiver compartment (PBS). Since water diffuses through human skin fairly readily, the osmotic pressure will drive water from the receiver into the salt rich donor compartment. We observed that lidocaine crystals precipitate from the donor compartment as it becomes infused with water. This reverse gradient could explain the reduced lidocaine transport from a saturated NMP solution. A rationale for the synergy of the IPM/NMP cosolvent system is the hydrophobic nature of IPM. Since IPM is not miscible with water, its presence in the donor compartment might deter the flux of water across the skin. In the absence of this osmotic pressure effect (2% lidocaine load), NMP shows improved transport properties. At this concentration, the permeability is over 25-fold better than saturated NMP.

At low drug concentration (2% lidocaine), a different IPM/NMP interaction likely occurs (FIG. 16). Below a threshold of ˜10% NMP, there is little effect on drug flux enhancement. Above this concentration, NMP permeability reaches a high level, and remains constant in the 10% to 75% NMP range. Increasing NMP beyond this level quickly diminishes permeability. The reason for this is unclear, but may stem from molecular interactions between NMP, IPM, and lidocaine. A possible hypothesis is that when NMP is close to 100% of the solution, it forms solvent-solvent bonds which make leaving the donor compartment unfavorable. When the non-polar IPM is introduced, it disrupts these interactions and pushes the NMP equilibrium into the donor. The exact mechanism is an avenue of further research.

From our experiments, we conclude that NMP is the preferred transdermal enhancer. Its method of action is most likely through improving the partitioning of lidocaine free base through the SC barrier. This process may be facilitated by hydrogen bonding between NMP and the drug, as suggested by IR spectroscopy. Experimentally, there was strong correlation between NMP flux and lidocaine flux. This raises the possibility that NMP may act as a “molecular chaperone” to enhance drug delivery. NMP displays very high permeability through human SC (1.8•10⁻² cm hr⁻¹ in the NMP/IPM systems), which may serve as a driving force for lidocaine free base flux. This same property should also apply to other drugs which hydrogen bond with NMP.

F. Tables

TABLE 5
Effect of Chemical Enhancers on Lidocaine Transport

| | | Stripped Human Cadaver Skin | | Full Thickness Pig Skin | |
|---|---|---|---|---|---|
| | | | Time Averaged | | Time Averaged |
| | Saturation | Time Averaged | Flux | Time Averaged | Flux |
| | Concentration | Permeability | (μg · cm⁻² · hr⁻¹) ± | Permeability | (μg · cm⁻² · hr⁻¹) ± |
| Sample | (mg/ml) | (cm hr⁻¹ · 10⁵) ± SD | SD | (cm hr⁻¹ · 10⁵) ± SD | SD |
| H₂O | 4.5 | 238 ± 43 | 10.8 ± 1.95 | 6.93 ± 3.46 | 0.496 ± 0.248 |
| 1% SDS | 8.3 | 246 ± 168 | 21.3 ± 14.6 | 9.29 ± 3.60 | 0.804 ± 0.312 |
| in H₂O | | | | | |
| IPM | 246 | 7.17 ± 1.06 | 20.4 ± 3.02 | 0.515 ± 0.117 | 0.847 ± 0.192 |
| NMP | 733 | 2.92 ± 0.42 | 26.8 ± 3.86 | 0.120 ± 0.007 | 0.706 ± 0.043 |
| Oleyl | 361 | 3.68 ± 1.59 | 14.5 ± 6.28 | — | — |
| Alcohol | | | | | |
| Oleic Acid | 428 | 0.61 ± 0.28 | 4.04 ± 1.83 | — | — |
| Butene | 386 | 1.18 ± 0.47 | 4.56 ± 1.80 | — | — |
| Diol | | | | | |
| Ethanol | 618 | 5.14 ± 3.55 | 31.7 ± 21.9 | 0.0649 ± 0.0190 | 0.182 ± 0.053 |

TABLE 6
Effect of 1:1 Cosolvent Systems on Lidocaine Transport

| | | Stripped Human Cadaver Skin | | Full Thickness Pig Skin | |
|---|---|---|---|---|---|
| | | | Time Averaged | | Time Averaged |
| | Saturation | Time Averaged | Flux | Time Averaged | Flux |
| | Concentration | Permeability | (μg · cm⁻² · hr⁻¹) ± | Permeability | (μg · cm⁻² · hr⁻¹) ± |
| Sample | (mg/ml) | (cm hr⁻¹ · 10⁵) ± SD | SD | (cm hr⁻¹ · 10⁵) ± SD | SD |
| IPM | 246 | 7.17 ± 1.06 | 20.4 ± 3.02 | 0.515 ± 0.117 | 0.847 ± 0.192 |
| IPM/NMP | 365 | 20.0 ± 6.92 | 53.4 ± 18.5 | 1.37 ± 0.69 | 3.64 ± 1.83 |
| 9:1 | | | | | |
| IPM/NMP | 641 | 18.9 ± 3.10 | 165 ± 27.1 | 0.965 ± 0.521 | 2.78 ± 1.50 |
| 1:1 | | | | | |
| NMP | 733 | 2.92 ± 0.42 | 26.8 ± 3.86 | 0.120 ± 0.007 | 0.706 ± 0.043 |
| IPM/Oleyl | 345 | 3.62 ± 1.12 | 17.7 ± 5.48 | — | — |
| Alcohol | | | | | |
| IPM/Oleic | 355 | 4.93 ± 0.57 | 32.5 ± 3.73 | — | — |
| Acid | | | | | |
| IPM/Decanoic | 309 | 2.47 ± 1.50 | 7.64 ± 4.65 | — | — |
| Acid | | | | | |

TABLE 7
Lidocaine Transport from IPM/NMP Cosolvent Systems Through Stripped Human
Cadaver Skin

| | | Lidocaine | | NMP | |
|---|---|---|---|---|---|
| | | | 24 Hour | | 24 Hour |
| % NMP | NMP | Fluxₛₛ | Transport | Fluxₛₛ | Transport |
| (v/v) | (mg/ml) | (μg cm⁻² hr⁻¹) ± SD | (μg) ± SD | (μg cm⁻² hr⁻¹) ± SD | (μg) ± SD |
| 0 | 0 | 1.98 ± 0.57 | 31.4 ± 6.4 | 0 | 0 |
| 10 | 105 | 4.69 ± 0.84 | 99.7 ± 25.7 | 1.46 ± 0.9 | 22.8 ± 3.7 |
| 25 | 259 | 16.4 ± 0.5 | 443 ± 73 | 5.40 ± 0.49 | 83.6 ± 5.7 |
| 50 | 518 | 32.5 ± 4.0 | 731 ± 179 | 9.01 ± 0.84 | 112 ± 5.3 |
| 60 | 620 | 40.7 ± 1.2 | 729 ± 72 | 11.2 ± 1.2 | 134 ± 13 |
| 75 | 778 | 56.7 ± 4.9 | 1040 ± 50 | 14.0 ± 0.9 | 161 ± 6 |
| 90 | 935 | 57.6 ± 8.4 | 907 ± 2 | 11.7 ± 0.1 | 128 ± 1 |
| 100 | 1040 | 15.4 ± 0.6 | 333 ± 25 | 10.7 ± 0.2 | 115 ± 0.2 |

G. Conclusion

NMP is capable of enhancing transdermal delivery by chaperoning lidocaine across human skin. The maximum lidocaine flux occurs from a solution of 25:75 IPM/NMP. There is strong correlation between NMP flux and lidocaine flux across skin samples. It is hypothesized that NMP participates in drug transport via hydrogen bonding. The high lidocaine flux obtained from the IPM/NMP cosolvent is a promising indication of the utility of this vehicle for transdermal drug delivery.

H. References for Example 2

15. Smith, D W; Peterson, M R; DeBerard, S C. Postgraduate Medicine. 1999, 106(2), 57-60, 64-66
16. Sleight, P. J. Cardiovasc. Pharmacol. 1990, 16, Suppl 5: S113-119
17. Ranade, Vasant V. J. Clin. Pharmacol. 1991, 31, 401-418
18. Johnson, Mark E.; Blankschtein, Daniel; Langer, Robert. Journal of Pharmaceutical Sciences. 1997, 86, 1162-1172
19. Walters, K. A. In Penetration enhancers and their use in transdermal therapeutic systems; Hadgraft, J.; Guy, R.; Eds.; Marcel Dekker: New York, 1989, 197-246
20. Mitragotri, S.; Blankschtein, D.; Langer, R. Science. 1995, 269, 850-853
21. Burnette, R. R. In Iontophoresis; Hadgraft, J.; Guy, R. H.; Eds.; Marcel Dekker: New York, 1989; 247-291
22. Prausnitz, M. R.; Bose, V. G.; Langer, R.; Weaver, J. C. PNAS 1993, 90, 10504-10508
23. Peck, Kendall D.; Ghanem, Abdel-Halem; Higuchi, William I. J. Pharm. Sci. 1995, 84, 975-982
24. Francoeur, Michael L.; Golden, Guia M.; Potts, Russell O. Pharm. Res. 1990, 7, 621-627
25. Kim, Dae-Duk; Chien, Yie W. J. Pharm. Sci. 1996, 85, 214-219
26. Guy, Richard H.; Hadgraft, Jonathan. J. Controlled Release. 1987, 5, 43-51
27. Gorukanti, Sudhir R.; Li, Lianli; Kim, Kwon H. Int. J. Pharm. 1999, 192, 159-172
28. Liu, Puchun; Kurihara-Bergstrom, Tamie; Good, William R. Pharm. Res. 1991, 8, 938-944
29. Yoneto, Kunio; Li, S. Kevin; Ghanem, Abdel-Halim; Crommelin, Daan J. A.; Higuchi, William I. J. Pharm. Sci. 1995, 84, 853-860
30. Sarpotdar, Pramod P.; Zatz, Joel L. J. Pharm. Sci. 1986, 75, 176-181
31. Johnson, Mark E.; Mitragotri, Samir; Patel, Ashish; Blankschtein, Daniel; Langer, Robert. J. Pharm. Sci. 1996, 85, 670-679
32. Sasaki, Hitoshi; Kojima, Masaki; Nakamura, Junzo; Shibasaki, Juichiro. J. Pharm. Pharmacol. 1990, 42, 196-199
33. Johnson, Mark E., Blankschtein, Daniel; Langer, Robert. J. Pharm. Sci. 1995, 84, 1144-1146
34. Williams, A. C.; Cornwell, P. A.; Barry, B. W. Int. J. Pharm. 1992, 86, 69-77
35. Priborsky, Jan; Takayama, Kozo; Nagai, Tsuneji; Waitzova, Danuse; Elis, Jiri. Drug Design and Delivery. 1987, 2, 91-97
36. Lee, Cheon Koo; Uchida, Takahiro; Kitagawa, Kazuhisa; Yagi, Akira; Kim, Nak-Seo; Goto, Shigeru. J. Pharm. Sci. 1994, 83, 562-565
37. Mitragotri, Samir; Johnson, Mark E.; Blankschtein, Daniel; Langer, Robert. Biophysical Journal. 1999, 77, 1268-1283.
38. Mitragotri, Samir. Pharm. Res. 2001, 18(7), 1018-23
39. Phillips, Christine A.; Michniak, Bozena B. J. Pharm. Sci. 1995, 84, 1427-1433

### EXAMPLE 3

### Chaperone-Mediated Transport for Transdermal Delivery

The interaction between chemical enhancers in a transdermal formulation is crucial to its function. In this study, n-methyl pyrrolidone (NMP) was studied as a water phase enhancer for lidocaine free base transport. It has been proposed that NMP acts as a flux chaperone via hydrogen bonding with solutes. This paper supports this hypothesis by finding that lipid disrupting agents (LDAs) capable of hydrogen bonding with NMP provide better lidocaine free base flux than analogous non-hydrogen bonding molecules. It was also found that NMP is capable of the chaperone effect above 50% v/v in H₂O for lidocaine free base. Addition of the LDA oleic acid improved flux up to 6-fold in the presence of NMP (35.1 μg/cm²/hr). The H₂O/NMP (50% v/v) system increased the flux of the hydrophilic ionic salt drugs lidocaine HCl and prilocaine HCl 4.3 and 2.6 fold, respectively. These findings support the NMP chaperone hypothesis and suggest that NMP is capable of enhancing hydrophilic, ionic drugs from an aqueous solution.

A. Introduction

Transdermal drug delivery is a promising route for the administration of therapeutic agents to the bloodstream painlessly and in a controlled manner.^{40,41} Current methods which have been developed to improve transdermal transport include chemical enhancers,⁴² therapeutic and low frequency ultrasound,⁴³ iontophoresis,⁴⁴ and electroporation.⁴⁵ It is crucial to investigate passive chemical enhancer systems because once favorable chemical interactions are found, they can be applied to the other means of transdermal enhancement.⁴⁶ Theoretical frameworks have been proposed to explain the effects of molecular size,⁴⁷ diffusion,^{48,49} and partitioning^{50,51} across bilayer membranes. However, one of the more complex parameters affecting transdermal drug delivery is the interaction of the constituents in the enhancer formulation.⁵² Although combining chemical enhancers often results in greatly improved drug flux,^{53,54} the mechanism of this effect is often difficult to determine. This paper will focus on the chemical enhancer n-methyl pyrrolidone (NMP) as a flux chaperone as well as its role in an aqueous solvent.
⁴⁰Asbill, C S; El-Kattan, A F; Michniak, B. Crit. Rev. Ther. Drug Carrier Syst. 2000, 17(6), 621-58

⁴¹Ranade, Vasant V. J. Clin. Pharmacol. 1991, 31, 401-418

⁴²Walters, K. A. In Penetration enhancers and their use in transdermal therapeutic systems; Hadgraft, J.; Guy, R.; Eds.; Marcel Dekker: New York, 1989, 197-246

⁴³Mitragotri, S.; Blankschtein, D.; Langer, R. Science. 1995, 269, 850-853

⁴⁴Burnette, R. R. In Iontophoresis; Hadgraft, J.; Guy, R. H.; Eds.; Marcel Dekker: New York, 1989; 247-291

⁴⁵Prausnitz, M. R.; Bose, V. G.; Langer, R.; Weaver, J. C. PNAS 1993, 90, 10504-10508

⁴⁶Mitragotri, Samir. Pharm. Res. 2000, 17(11), 1354-58

⁴⁷Mitragotri, Samir; Johnson, Mark E.; Blankschtein, Daniel; Langer, Robert. Biophysical Journal. 1999, 77, 1268-1283.

⁴⁸Johnson, Mark E.; Blankschtein, Daniel; Langer, Robert. Journal of Pharmaceutical Sciences. 1997, 86, 1162-1172

⁴⁹Johnson, Mark E.; Berk, David A.; Blankschtein, Daniel; Golan, David E.; Jain, Rakesh K.; Langer, Robert. Biophysical Journal. 1996, 71, 2656-2668.

⁵⁰Phillips, Christine A.; Michniak, Bozena B. J. Pharm. Sci. 1995, 84, 1427-1433

⁵¹Lee, Cheon Koo; Uchida, Takahiro; Kitagawa, Kazuhisa; Yagi, Akira; Kim, Nak-Seo; Goto, Shigeru. J. Pharm. Sci. 1994, 83, 562-565

⁵²Pugh, W. J.; Hadgraft, J.; Roberts, M. S. Int. J. Pharm. 1996, 138, 149-65

⁵³Johnson, Mark E.; Mitragotri, Samir; Patel, Ashish; Blankschtein, Daniel; Langer, Robert. J. Pharm. Sci. 1996, 85, 670-679

⁵⁴Sasaki, Hitoshi; Kojima, Masaki; Nakamura, Junzo; Shibasaki, Juichiro. J. Pharm. Pharmacol. 1990, 42, 196-199

Although there is no broad theoretical model predicting solute-solvent interactions in transdermal flux, it has been shown in previous experiments that strong relationships can exist.^{55,56} Results across model membranes indicate that solute flux is proportional to solvent uptake. Furthermore, this parameter can be predicted based on solubility, MW, and hydrogen bonding interactions. Our previous work with NMP suggests that it is capable of enhancing lidocaine free base transport across human skin. It has been hypothesized that this is mediated through hydrogen bonding between NMP and the amide group of lidocaine free base. When in the presence of the highly lipophilic solvent isopropyl myristate (IPM), NMP exhibits significant enhancement properties for lidocaine free base transport. It was also found that the degree of drug flux correlated closely with the amount of NMP flux across the skin. This finding supported the hypothesis that NMP can act as a chaperone for transdermal transport across human skin. An important question addressed by the current study is whether NMP in a water phase solvent is capable of the same enhancement.
⁵⁵Liu, Puchun; Kurihara-Bergstrom, Tamie; Good, William R. Pharm. Res. 1991, 8, 938-944

⁵⁶Cross, Sheree; Pugh, W. John; Hadgraft, Jonathan; Roberts, Michael S. Pharm. Res. 2001, 18(7) 999-1005

An enhancer capable of improving drug flux from the water phase would greatly expand the current repertoire of transdermally deliverable drugs. The primary barrier to drug transport across human skin is the stratum corneum.⁵⁷ Due to the nature of this lipid membrane, aqueous phase transport has proven difficult.^{58,59,60} NMP was investigated as a possible water phase flux enhancer due to its miscibility with water and earlier reports of improved transdermal delivery.^{61,62,63} Additionally, based on the hypothesis of hydrogen bonding between NMP and formulation solutes as a means of enhancement, the effect of lipid disrupting agents (such as oleic acid)^{64,65} should also be aided by NMP. The flux enhancement of analogous 18 carbon molecules with different end groups (and thus differing hydrogen bonding capacity) was used to test this hypothesis. Lidocaine free base was selected as the model drug as its interaction with NMP has been previously studied. It can also be derived as a water soluble, ionic salt (lidocaine HCl). NMP systems were further investigated to determine whether they are capable of providing enhancement of the hydrophilic ionic drugs lidocaine HCl and prilocaine HCl.
⁵⁷Barry, B. W.; Bennett, S. L. J. Pharm. Pharmacol. 1987, 39, 535-46

⁵⁸Gorukanti, Sudhir R.; Li, Lianli; Kim, Kwon H. Int. J. Pharm. 1999, 192, 159-172

⁵⁹Roy, Samir D.; Roos, Eric; Sharma, Kuldeepak. J. Pharm. Sci. 1994, 83, 126-130

⁶⁰Peck, Kendall D.; Ghanem, Abdel-Halem; Higuchi, William I. J. Pharm. Sci. 1995, 84, 975-982

⁶¹Sasaki, Hitoshi; Kojima, Masaki; Nakamura, Junzo; Shibasaki, Juichiro. J. Pharm. Pharmacol. 1990, 42, 196-199

⁶²Phillips, Christine A.; Michniak, Bozena B. J. Pharm. Sci. 1995, 84, 1427-1433

⁶³Yoneto, Kunio; Li, S. Kevin; Ghanem, Abdel-Halim; Crommelin, Dann J. A.; Higuchi, William I. J. Pharm. Sci. 1995, 84(7), 853-61

⁶⁴Francoeur, Michael L.; Golden, Guia M.; Potts, Russell O. Pharm. Res. 1990, 7, 621-627

⁶⁵Kim, Dae-Duk; Chien, Yie W. J. Pharm. Sci. 1996, 85, 214-219

B. Materials

Drugs: Lidocaine free base, lidocaine HCl, and prilocaine HCl were purchased from Sigma (St. Louis, Mo.). Chemicals: NMP was a generous gift from ISP Technologies, Inc. (Wayne, N.J.). USP grade oleic acid was purchased from Mednique. Isopropyl myristate (IPM), 9-octadecene, oleyl alcohol (99%), anhydrous ethyl alcohol, and phosphate buffered saline tablets (PBS) were purchased from Sigma (St. Louis, Mo.). HPLC grade solvents were used as received. Skin: Human cadaver skin from the chest, back, and abdominal regions was obtained from the National Disease Research Institute (Philadelphia, Pa.). The skin was stored at −80° C. until use.

C. Methods

(i) Preparation of Drug Solutions. Sample solutions were prepared in 20 ml glass vials and loaded with drug. All vehicles studied formed miscible, single phase liquids.

(ii) Preparation of Skin Samples. Human cadaver skin was thawed at room temperature. The epidermis-SC was separated from the full thickness tissue after immersion in 60° C. water for 2 minutes. Heat stripped skin was immediately mounted on diffusion cells.

(iii) Transport Experiments. The skin was mounted onto a side-by-side glass diffusion cell with an inner diameter of 5 mm. The two halves of the cell were clamped shut and both reservoirs were filled with 2 ml of phosphate buffered saline (PBS, 0.01 M phosphate, 0.137 M NaCl, pH 7.4). The integrity of the skin was verified by measuring the electrical conductance across the skin barrier at 1 kHz and 10 Hz at 143.0 mV (HP 33120A Waveform Generator). Skin samples measuring 4-10 μA at 1 kHz were used for the diffusion studies. Prior to introducing the donor solution, the skin sample was thoroughly rinsed with PBS to remove surface contaminants. At t=0, the receiver compartment was filled with 2.0 ml of PBS, while 2.0 ml of sample was added to the donor compartment. Both compartments were continuously stirred to maintain even concentrations. At regular time intervals, 1.0 ml of the receiver compartment was transferred to a glass HPLC vial. The remaining solution in the receiver compartment was thoroughly aspirated and discarded. Fresh PBS (2.0 ml) was dispensed into the receiver compartment to maintain sink conditions. At 24 hours, the experiment was terminated. After both compartments were refilled with PBS, the conductance across the skin membrane was again checked to ensure that the skin was not damaged during the experiment. All flux experiments were conducted in triplicate at room temperature. The observed variability of the individual drug transport values was consistent with the previously established 40% intersubject variability of human skin.⁶⁶
⁶⁶Williams, A. C.; Cornwell, P. A.; Barry, B. W. Int. J. Pharm. 1992, 86, 69-77

(iv) LDA Chaperoning. IPM solutions of 2% (w/v) lidocaine free base and 1% (w/v) LDA were mixed in 20 ml glass vials. The LDA's included octadecene, oleyl alcohol, and oleic acid. Similar solutions with NMP as the solvent were also obtained. The flux of lidocaine free base and of NMP through human cadaver skin was determined as detailed above.

(v) H₂O/NMP Binary Vehicle Transport. The two miscible liquids were mixed in the specified v/v ratios, with 2% w/v lidocaine free base added. Flux cells were set up as described above. At t=20, 22, 24 hours, the transport of drug across the skin was measured by HPLC. Steady state conditions were taken as the average of the final 2 time points. After 24 hours, the drug solution was removed and the skin rinsed with PBS. Two ml of the equivalent H₂O/NMP mixture with 1% oleic acid (v/v) was then added to the donor compartment of the flux cell. The effect of this solution on drug transport was measured by HPLC after an additional 6, 7, and 8 hours.

(vi) Hydrophilic Drug Transport The water soluble drugs lidocaine HCl and prilocaine HCl were dissolved in 2% (w/v) doses in 1:1 H₂O:NMP (v/v). Flux through stripped human skin was compared with flux of the same drugs through distilled water. After 24 hours, oleic acid (1%) was introduced to the donor solutions of the lidocaine HCl samples. All drug concentrations were analyzed by HPLC.

(vii) NMP Patitioning. Distilled water (2 ml), IPM (2 ml), and NMP (40 μl) were thoroughly vortexed in a glass tube. After equilibrating for 1 hour, the sample was centrifuged at 14,000 rpm for 6 minutes and separated into 2 phases. Samples of each phase were taken to determine NMP concentration by HPLC.

(viii) Quantification of Transport. Lidocaine was assayed by high pressure liquid chromatography (Shimadzu model HPLC, SCL-10A Controller, LC-10AD pumps, SPD-M10A Diode Array Detector, SIL-10AP Injector, Class VP v.5.032 Integration Software) on a reverse phase column (Waters μBondapak™ C₁₈ 3.9×150 mm) using ddH₂O (5% acetic acid, pH 4.2)/acetonitrile (35:65 v/v) as the mobile phase, under isocratic conditions (1.6 mL/min) by detection at 237 nm. The retention time of lidocaine under these conditions was between 3.4 and 4.3 minutes. Standard solutions were used to generate calibration curves. The same HPLC method was utilized for prilocaine HCl, with the exception that it was measured at 254 nm. NMP was quantified on a Waters Symmetry® C₁₈ 5 μm, 3.9×150 mm column (WAT046980). The mobile phase consisted of ddH₂O:methanol (95:5) at a flow rate of 1.2 ml/min. Chromatograms were integrated at a peak of 205 nm, with retention time at 3.8-4.8 min.

(ix) Calculations. The total mass of drug transported across the skin was determined by HPLC. The flux equation gives:

$$J = \frac{1}{A} ( \frac{ⅆ M}{ⅆ t} ) = P Δ C$$

where J is flux (μg cm⁻² hr⁻¹), A is cross sectional area of the skin membrane (cm²), P is the apparent permeability coefficient (cm hr⁻¹), and ΔC is the concentration gradient. In this experiment, ΔC is taken as the donor concentration (assuming infinite dose and sink conditions), and dM/dt is averaged as the total mass transport over the time course of the final two time points. Statistical analyses were performed by the Student's t-test.
D. Results

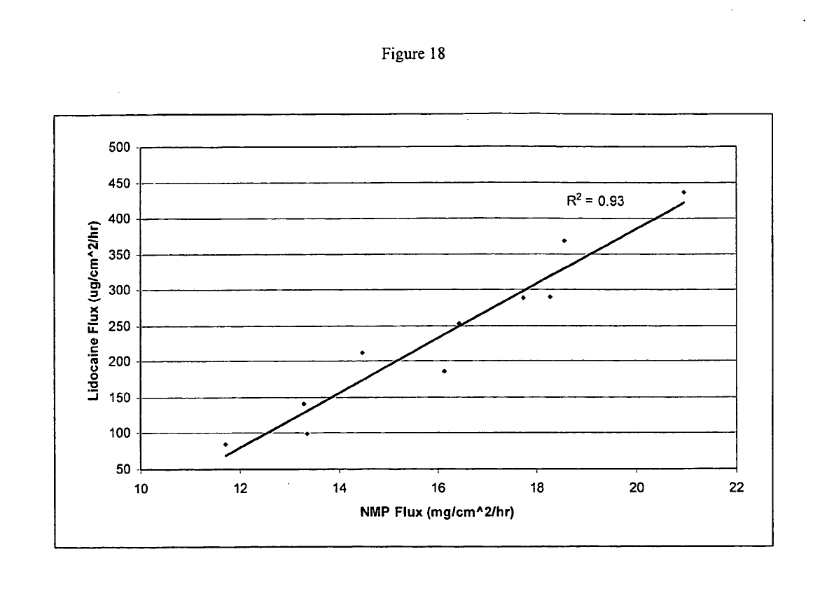

(i) LDA Chaperoning. Flux of lidocaine free base through human cadaver skin from IPM solutions is given in Table 8. There is no statistical significance (p>0.40) among any of the IPM samples. When NMP is used as the bulk solvent, there is a trend of increasing lidocaine free base flux from no LDA, octadecene, IPM, oleic acid, oleyl alcohol. All four lipid compounds improve drug flux over neat NMP (p<0.05). The best enhancer, oleyl alcohol is statistically better than IPM and octadecene (p<0.05). The flux of NMP from these solutions closely matches that of lidocaine transport, with a R² value of 0.93 (FIG. 18).

(ii) H₂O/NMP Binary Cosolvent. Lidocaine free base (2% w/v) transport was investigated in varying combinations of H₂O/NMP (Table 9). Because lidocaine free base is sparsely soluble in water, the 80%, 90%, and 100% H₂O samples were saturated below 2% drug. Both the flux and permeability of drug at varying NMP concentration results in a V-shaped curve. NMP does not begin transporting across the skin unless it is above ˜50% of the donor solution. Plotting the NMP and lidocaine fluxes for % NMP≧50% results in a strong correlation (R² value of 0.98). When 1% oleic acid is used, the flux of the hydrophobic drug increases. The samples with greater NMP flux (in the absence of oleic acid) showed improved enhancement when oleic acid is introduced. Specifically, at 40% NMP (poor NMP flux), oleic acid had no effect (p>0.5) while at 80% NMP the flux enhancement was over 6-fold (35.1 μg/cm²/hr).

(iii) Hydrochloride Salt Transport. The flux of the hydrochloride salt drugs lidocaine HCl and prilocaine HCl were investigated in H₂O/NMP (50% v/v). NMP appears to be capable of improving the flux of the two drugs roughly 2-4 fold (Table 10). The addition of 1% oleic acid to H₂O/NMP in does not affect lidocaine HCl flux (p>0.5). When an IPM/NMP (50% v/v) system was utilized, the lidocaine HCl flux was even greater (15.7±7.9 μg/cm²/hr). This was accompanied by a significantly larger NMP flux through the skin.

(iv) IPM/H₂O Partitioning of NMP. The relative concentration of NMP was determined from an equilibrium mixture of H₂O and IPM. NMP was found to partition 98% in the water phase (IPM/H₂O=0.02±0.001).

E. Discussion

The role of NMP as an aqueous phase transdermal chemical enhancer was studied in this experiment. We tested one hypothesis that NMP acts as a chaperone molecule, facilitating solute transport into and across human skin via hydrogen bonding. Interactions among formulation components is an important field of study in transdermal drug delivery. Although some research has been done regarding hydrogen bonding between solutes and artificial membranes,⁶⁷ flux enhancement as a result of hydrogen bonding between two co-transported species is not well understood.
⁶⁷Du Plessis, Jeanetta; Pugh, W. John; Judefeind, Anja; Hadgraft, Jonathan. Eur. J. Pharm. Sci. 2001, 13, 135-141

It is proposed that NMP is capable of improving the efficacy of enhancing agents such as LDAs. Based on molecular structures, oleic acid and oleyl alcohol should have greater hydrogen bonding capacity with NMP than octadecene and IPM. The free hydroxyl groups of these two molecules are capable of hydrogen bonding with the NMP oxygen. Table 8 indicates that all 4 lipid disrupting-like agents have statistically equivalent effects on lidocaine free base transport from an IPM solvent. From these results, it is clear that none of these chemicals have an enhancing effect on lidocaine flux from the hydrophobic solvent. However, if the same agents are used in conjunction with an NMP solvent, there is a definite enhancing effect. The addition of each of the LDA-like molecules (1% v/v) improve drug flux over neat NMP solution (p<0.05). It is also apparent that both lidocaine free base and NMP flux from these systems follows the trend predicted by hydrogen bonding, with flux from oleic acid and oleyl alcohol systems being greater than IPM or octadecene. This suggests that from an IPM solution, the hydrophobic LDAs are incapable of affecting the skin membrane. One possible explanation is that the LDAs have such high affinity for the donor solvent that they do not partition into the skin. Once they are in the environment of a polar solvent (NMP), the LDAs are capable of partitioning into the hydrophobic stratum corneum. When present in the skin, the lipid disrupting agents are thought to reduce the barrier properties of the stratum corneum, and improve drug permeability by creating disorder in the lipids.²⁵ The subsequent enhancement in both NMP flux and lidocaine free base flux can be explained by this effect of the LDAs.
²⁵Kim, Dae-Duk; Chien, Yie W. J. Pharm. Sci. 1996, 85, 214-219

Both NMP and the LDAs enhance the transport of each of the other components in the formulation, resulting in significantly improved drug flux. Although the exact mechanism is not clear, the results from this experiment can be explained in the following manner. The LDAs are in an environment favorable to their partitioning into the skin. As NMP has a high inherent flux through skin (12.6±0.5 mg/cm²/hr), it is able to facilitate the transport of the LDA into the skin via the hypothesis of hydrogen bonding. This, in turn, reduces the barrier properties of the skin, and improves NMP and drug flux. As NMP flux increases, it will cause an increase in drug flux due to its proposed chaperoning ability. This is supported by the high correlation (r²=0.93) between NMP and lidocaine free base flux in the formulations. Even with the large differences in LDAs and drug flux across the samples, there exists a tight relationship between the flux of NMP and drug, supporting the claim that there is indeed an interaction between the two molecules.

When NMP was used in conjunction with a water solvent, the results differed from that obtained with IPM/NMP. Two important characteristics of the synergy curve are markedly different: the magnitude and shape. The steady state flux of lidocaine free base from an H₂O/NMP system is significantly lower than an IPM/NMP system. The finding that NMP flux is lower from water than from IPM is not surprising. Because NMP partitions almost exclusively into the water phase from an IPM mixture ([IPM]/[H₂O]=0.02), it is much more likely to leave a donor solution of IPM into a receiver compartment of PBS than to partition from water to PBS. In the water phase, NMP flux is driven only by a concentration gradient. However, when IPM is the bulk solvent, the partitioning of NMP serves as an additional driving force.

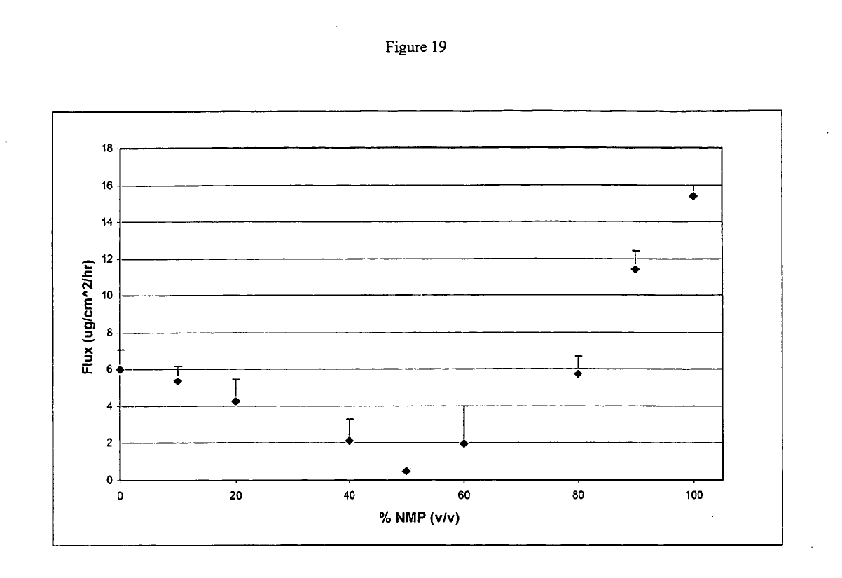

From a water phase system (Table 9), it appears that NMP has essentially zero flux out of the formulation until it reaches a concentration of about 50% (v/v). This might be explained by the fact that NMP has very high affinity for water. In small quantities, NMP may be completely solvated by water and thermodynamically unable to partition into the hydrophobic skin membrane. Even at 50% (v/v) NMP, there is still a 6:1 molar excess of water molecules in the system. Only when NMP exists at suitable concentrations is it free to transport across the skin. From 50-100% NMP, there is a steady increase in NMP flux across the skin (FIG. 19). In this range, it is observed that lidocaine free base flux also increases proportionally to NMP flux (r²=0.98). This supports the hypothesis that NMP acts as a drug chaperone. At and below 50% NMP, when its flux is negligible, there is no enhancement of lidocaine flux. In fact, the data seems to support that from 0-50% (v/v) NMP, lidocaine flux is retarded by increasing NMP. Lidocaine free base has a high permeability from water (13.3±2.3•10⁻⁴ cm/hr), and the addition of NMP reduces this value by 98% (0.24±0.05•10⁻⁴ cm/hr) at a concentration of 50% NMP. The interaction of NMP with water limits the permeability of lidocaine free base across human skin. A possible explanation of this result is that in the absence of NMP flux across the skin, the hydrophobic lidocaine free base molecules have increased affinity for the donor phase (with NMP as a solvent) and are less likely to partition into the skin. Further evidence of the NMP chaperone hypothesis is given by the observation that oleic acid is ineffective as an enhancing agent unless there is significant NMP flux through the skin. This is consistent with the finding in Table 8 where the absence of NMP flux renders the LDA ineffective.

Although the H₂O/NMP system is not as beneficial as IPM/NMP in improving the flux of the model drug lidocaine free base, its transport does support the hypothesis that NMP is capable of acting as a chaperone in the water phase. This claim is further supported by the transport of the hydrophilic drugs lidocaine HCl and prilocaine HCl from the H₂O/NMP system. Both of these drugs are highly water soluble ionic compounds, making transdermal transport difficult.⁶⁸ From the data (Table 10), it appears that H₂O/NMP is capable of providing some flux enhancement for these drugs. Interestingly, when oleic acid (1% v/v) was added to the lidocaine HCl sample, the flux was unaffected (p>0.5). A possible hypothesis suggested by this observation is that the transport of lidocaine HCl goes through a pathway which is not limited by lipid bilayers. If this is the case, then NMP may be able to act as an enhancer in the aqueous transport pathway as well as the more commonly studied lipid route. Although the extent of this enhancement may be improved in numerous was, the finding that NMP can be effective in improving the delivery of hydrophilic, ionic drugs opens up a wide area of investigation.
⁶⁸Peck, Kendall D.; Ghanem, Abdel-Halem; Higuchi, William I. J. Pharm. Sci. 1995, 84, 975-982

F. Tables

TABLE 8
Lidocaine and NMP Flux with LDA

| | IPM Solvent | NMP Solvent | |
|---|---|---|---|
| | Lidocaine Fluxₛₛ | Lidocaine Fluxₛₛ | NMP Fluxₛₛ |
| LDA (1% w/v) | (μg/cm²/hr ± SD) | (μg/cm²/hr ± SD) | (mg/cm²/hr ± SD) |
| None | 1.95 ± 0.22 | 92 ± 15 | 12.6 ± 0.5 |
| Octadecene | 1.98 ± 0.58 | 161 ± 52 | 15.3 ± 1.2 |
| Isopropyl | — | 232 ± 120 | 15.8 ± 3.6 |
| Myristate | | | |
| Oleic Acid | 1.68 ± 0.24 | 290 ± 103 | 17.6 ± 2.0 |
| Oleyl Alcohol | 1.97 ± 0.48 | 402 ± 52 | 20.7 ± 1.0 |

N = 3

TABLE 9
Transport of Lidocaine and NMP from H₂O/NMP Binary Systems

| | Lidocaine Free Base | | NMP | |
|---|---|---|---|---|
| | Fluxₛₛ | Permeability | Fluxₛₛ | |
| | (μg/cm²/ | (cm/hr · | (μg/cm²/hr ± | Permeability (cm/ |
| % NMP | hr ± SD) | 10⁴ ± SD) | SD) | hr · 10⁴ ± SD) |
| 0 | 6.02 ± 1.04 | 13.3 ± 2.3 | — | — |
| 10 | 5.36 ± 0.83 | 5.43 ± 0.84 | 0.007 ± 0.007 | 1.08 ± 1.08 |
| 20 | 4.27 ± 1.20 | 2.86 ± 0.80 | 0.008 ± 0.003 | 0.64 ± 0.23 |
| 40 | 2.15 ± 1.13 | 1.08 ± 0.56 | 0.026 ± 0.021 | 1.01 ± 0.83 |
| 50 | 0.47 ± 0.10 | 0.24 ± 0.05 | 0.010 ± 0.002 | 0.31 ± 0.05 |
| 60 | 1.95 ± 2.07 | 0.97 ± 1.03 | 0.106 ± 0.058 | 2.73 ± 1.48 |
| 80 | 5.75 ± 0.95 | 2.87 ± 0.48 | 3.26 ± 0.74 | 62.8 ± 14.2 |
| 90 | 11.4 ± 1.0 | 5.71 ± 0.51 | 9.18 ± 0.53 | 157 ± 9 |
| 100 | 15.4 ± 0.6 | 7.69 ± 0.29 | 12.6 ± 0.5 | 63.1 ± 2.5 |

N = 3

TABLE 10
Flux Enhancement of Hydrophilic HCl Salt Drugs from 1:1
H2O/NMP Cosolvent through Stripped Human Cadaver Skin

| | | Fluxₛₛ from | |
|---|---|---|---|
| | Fluxₛₛ from H₂O | H₂O/NMP | |
| Drug (2% w/v) | (μg/cm²/hr ± SD) | (μg/cm²/hr ± SD) | Enhancement |
| Lidocaine HCl | 1.00 ± 0.22 | 4.35 ± 1.84 | 4.3 ± 2.1 |
| Prilocaine HCl | 2.44 ± 0.98 | 6.31 ± 0.14 | 2.6 ± 1.0 |

N = 3

G. Conclusion

This paper supports the claim that NMP acts as a transdermal enhancer through its hydrogen bonding capability with other formulation solutes. NMP was found to act in synergy with LDAs capable of hydrogen bonding (such as oleic acid and oleyl alcohol) to improve lidocaine free base flux. These same LDAs had no effect from IPM and H₂O solutions, suggesting that the presence of NMP is central to their enhancement ability. More specifically, it is crucial that NMP flux from the system be appreciable for LDA effectiveness. The enhancement of oleic acid on lidocaine free base flux was negligible from H₂O/NMP systems where there was no NMP flux (Table 9) as well as from IPM/NMP systems (˜10% NMP) where NMP flux was minimal (data not shown).

NMP also appears to be capable of providing drug delivery enhancement from the aqueous phase. In this paper, H₂O/NMP systems resulted in improved LDA (oleic acid) effect, hydrophobic drug flux (lidocaine free base), and hydrophilic ionic salt drug flux (lidocaine HCl and prilocaine HCl). All of these results are consistent with the hypothesis that NMP behaves as a transdermal chaperone, acting through its hydrogen bonding capacity and high flux through the skin.

H. References for Example 3

40. Asbill, C S; El-Kattan, A F; Michniak, B. Crit. Rev. Ther. Drug Carrier Syst. 2000, 17(6), 621-58
41. Ranade, Vasant V. J. Clin. Pharmacol. 1991, 31, 401-418
42. Walters, K. A. In Penetration enhancers and their use in transdermal therapeutic systems; Hadgraft, J.; Guy, R.; Eds.; Marcel Dekker: New York, 1989, 197-246
43. Mitragotri, S.; Blankschtein, D.; Langer, R. Science. 1995, 269, 850-853
44. Burnette, R. R. In Iontophoresis; Hadgraft, J.; Guy, R. H.; Eds.; Marcel Dekker: New York, 1989; 247-291
45. Prausnitz, M. R.; Bose, V. G.; Langer, R.; Weaver, J. C. PNAS 1993, 90, 10504-10508
46. Mitragotri, Samir. Pharm. Res. 2000, 17(11), 1354-58
47. Mitragotri, Samir; Johnson, Mark E.; Blankschtein, Daniel; Langer, Robert. Biophysical Journal. 1999, 77, 1268-1283.
48. Johnson, Mark E.; Blankschtein, Daniel; Langer, Robert. Journal of Pharmaceutical Sciences. 1997, 86, 1162-1172
49. Johnson, Mark E.; Berk, David A.; Blankschtein, Daniel; Golan, David E.; Jain, Rakesh K.; Langer, Robert. Biophysical Journal. 1996, 71, 2656-2668.
50. Phillips, Christine A.; Michniak, Bozena B. J. Pharm. Sci. 1995, 84, 1427-1433
51. Lee, Cheon Koo; Uchida, Takahiro; Kitagawa, Kazuhisa; Yagi, Akira; Kim, Nak-Seo; Goto, Shigeru. J. Pharm. Sci. 1994, 83, 562-565
52. Pugh, W. J.; Hadgraft, J.; Roberts, M. S. Int. J. Pharm. 1996, 138, 149-65
53. Johnson, Mark E.; Mitragotri, Samir; Patel, Ashish; Blankschtein, Daniel; Langer, Robert. J. Pharm. Sci. 1996, 85, 670-679
54. Sasaki, Hitoshi; Kojima, Masaki; Nakamura, Junzo; Shibasaki, Juichiro. J. Pharm. Pharmacol. 1990, 42, 196-199
55. Liu, Puchun; Kurihara-Bergstrom, Tamie; Good, William R. Pharm. Res. 1991, 8, 938-944
56. Cross, Sheree; Pugh, W. John; Hadgraft, Jonathan; Roberts, Michael S. Pharm. Res. 2001, 18(7) 999-1005
57. Barry, B. W.; Bennett, S. L. J. Pharm. Pharmacol. 1987, 39, 535-46
58. Gorukanti, Sudhir R.; Li, Lianli; Kim, Kwon H. Int. J. Pharm. 1999, 192, 159-172
59. Roy, Samir D.; Roos, Eric; Sharma, Kuldeepak. J. Pharm. Sci. 1994, 83, 126-130
60. Peck, Kendall D.; Ghanem, Abdel-Halem; Higuchi, William I. J. Pharm. Sci. 1995, 84, 975-982
61. Sasaki, Hitoshi; Kojima, Masaki; Nakamura, Junzo; Shibasaki, Juichiro. J. Pharm. Pharmacol. 1990, 42, 196-199
62. Phillips, Christine A.; Michniak, Bozena B. J. Pharm. Sci. 1995, 84, 1427-1433
63. Yoneto, Kunio; Li, S. Kevin; Ghanem, Abdel-Halim; Crommelin, Dann J. A.; Higuchi, William I. J. Pharm. Sci. 1995, 84(7), 853-61
64. Francoeur, Michael L.; Golden, Guia M.; Potts, Russell O. Pharm. Res. 1990, 7, 621-627
65. Kim, Dae-Duk; Chien, Yie W. J. Pharm. Sci. 1996, 85, 214-219
66. Williams, A. C.; Cornwell, P. A.; Barry, B. W. Int. J. Pharm. 1992, 86, 69-77
67. Du Plessis, Jeanetta; Pugh, W. John; Judefeind, Anja; Hadgraft, Jonathan. Eur. J. Pharm. Sci. 2001, 13, 135-141
68. Peck, Kendall D.; Ghanem, Abdel-Halem; Higuchi, William I. J. Pharm. Sci. 1995, 84, 975-982

### EXAMPLE 4

### Microemulsion Enhancer Formulation for Simultaneous Transdermal Delivery of Hydrophilic and Hydrophobic Drugs

Microemulsion (ME) systems allow for the microscopic incorporation of aqueous and organic phase liquids. In this study, the phase diagrams of four novel ME systems were characterized. Water and IPM composed the aqueous and organic phases respectively, while Tween 80 served as an anionic surfactant. Transdermal enhancers such as n-methyl pyrrolidone (NMP) and oleyl alcohol were incorporated into all systems without disruption of the stable emulsion. A comparison of a W/O ME with an O/W ME of the same system for lidocaine delivery indicated that an O/W ME provides significantly greater flux (p<0.025). This finding, that the water phase is a crucial component is consistent with in vitro flux experiments using hydrophobic drugs (lidocaine free base, estradiol) as well as hydrophilic drugs (lidocaine HCl, diltiazem HCl). Furthermore, the simultaneous delivery of both a hydrophilic drug and a hydrophobic drug from the ME system is indistinguishable from either drug alone. Enhancement of drug permeability from the O/W ME system was 17-fold for lidocaine free base, 30-fold for lidocaine HCl, 58-fold for estradiol, and 520-fold for diltiazem HCl.

A. Introduction

Microemulsions (ME) are thermodynamically stable emulsions with droplet sizes in the sub-micron range. They typically consist of an aqueous phase, an organic phase, and a surfactant/cosurfactant component. The design and properties of microemulsion systems is a field that has been studied extensively with applications in many pharmaceutical areas.⁶⁹ There are two basic types of ME systems: water-in-oil (W/O) and oil-in-water (O/W). In each case, it is believed that the minority phase is encapsulated by the continuous bulk phase. Surfactants are necessary to reduce the hydrophobic interactions between the phases and maintain a single phase. Typical properties of ME include optical transparency, thermodynamic stability, and solubility of both hydrophobic and hydrophilic components.
⁶⁹Lawrence, M. J., et. al. Int. Journal of Pharmaceutics. 1998, 111, 63-72

Microemulsions have been proposed to offer enhanced drug delivery properties for transdermal transport.^{70,71} Flux enhancement from these formulations was found to be primarily due to an increase in drug concentration. In these studies, it was concluded that drug transport occurs only from the continuous (outer) phase. By this account, hydrophobic drugs transport faster from W/O emulsions, while O/W systems provide slower, controlled release of drug that is dependant on the partitioning of drug into the outer phase. This pathway of drug release from ME systems is supported by work with a hydrophilic molecule (glucose) where it was found to parallel the diffusion of water from the bulk phase.⁷² The stability and encapsulation properties of emulsions make the transdermal delivery of protein drugs an ideal application.^{73,74,75,76}
⁷⁰Trotta, M.; Gasco, M. R.; Morel, S. Journal of Controlled Release. 1989, 10, 237-243

⁷¹Trotta, M.; Pattarino, F.; Gasco, M. R. Pharmaceutica Acta Helvetiae. 1996, 71, 135-140

⁷²Osborne, D. W.; Ward, A. J. I.; O'Neill, K. J. J. Pharm. Pharmacol. 1991, 43, 451-454

⁷³Ho, Hsiu-O; Hsiao, Chih-Chuan; Sheu, Ming-Thau. J. Pharm. Sci. 1996, 85(2) 138-143

⁷⁴Guo, Jianxin; Ping, Qineng; Sun, Guoqin; Jiao, Chunhong. Int. J. Pharm. 2000, 194, 201-207

⁷⁵Zhang, Qiang; Yie, Guoqing; Li, Yie; Yang, Qingsong; Nagai, T. Int. J. Pharm. 2000, 200, 153-159

⁷⁶Baca-Estrada, Maria E.; Foldvari, Marianna; Ewen, Catherine; Badea, Ildiko; Babiuk, Lorne A. Vaccine. 2000, 18, 1847-1854

In this study, multiple features were incorporated into a ME formulation. Nonionic surfactants were selected to minimize skin irritation and charge disruption of the system. The main surfactant studied, Tween 80 (Polysorbate 80) has previously been utilized in transdermal formulations.^{77,78} A key feature of the ME systems studied is incorporation of the transdermal chemical enhancers oleyl alcohol and n-methyl pyrrolidone (NMP), which to our knowledge has never been explored. Oleyl alcohol is a cis-unsaturated C₁₈ fatty acid which is believed to reduce the barrier properties of the skin by disrupting lipid bilayers within the stratum corneum.^{79,80} NMP has been utilized as a transdermal enhancer for multiple drugs and formulation compositions, but never in conjunction with a ME.^{81,82,83} We selected NMP based on our earlier studies showing that it is capable of significantly enhancing drug transport from both the organic⁸⁴ and aqueous⁸⁵ phase. These findings supported our hypothesis that the hydrogen bonding capability of NMP with certain drugs, along with the high flux of NMP through human skin (˜10 mg/cm²/hr) allows NMP to act as a molecular chaperone. We propose that this enhancing ability should occur in ME systems as well.
⁷⁷Walters, K. A; Dugard, P. H., Florence, A. T. J. Pharm. Pharmacol. 1981, 33, 207-213

⁷⁸Sarpotdar, Pramod P.; Zatz, Joel L. J. Pharm. Sci. 1986, 75, 176-181

⁷⁹Francoeur, Michael L.; Golden, Guia M.; Potts, Russell O. Pharm. Res. 1990, 7, 621-627

⁸⁰Kim, Dae-Duk; Chien, Yie W. J. Pharm. Sci. 1996, 85, 214-219

⁸¹Sasaki, Hitoshi; Kojima, Masaki; Nakamura, Junzo; Shibasaki, Juichiro. J. Pharm. Pharmacol. 1990, 42, 196-199

⁸²Phillips, Christine A.; Michniak, Bozena B. J. Pharm. Sci. 1995, 84, 1427-1433

⁸³Yoneto, Kunio; Li, S. Kevin; Ghanem, Abdel-Halim; Crommelin, Dann J. A.; Higuchi, William I. J. Pharm. Sci. 1995, 84(7), 853-61

⁸⁴Lee, Philip J.; Ahmad, Naina; Mitragotri, Samir; Langer, Robert, Shastri, V. Prasad. “Evaluation of Chemical Enhancers in the Transdermal Delivery of Lidocaine.” Submitted Pharm. Res.

⁸⁵Lee, Philip J.; Mitragotri, Samir; Langer, Robert, Shastri, V. Prasad. “Chaperoning of Lipid Disrupting Agents and Aqueous Phase Transdermal Enhancement by n-Methyl Pyrrolidone.” Submitted Pharm. Res.

In this study we have evaluated the transdermal transport of several hydrophobic and hydrophilic drug moieties from novel ME systems that incorporate chemical enhancers. Drug molecules investigated include lidocaine free base^{86,87} and HCl salt, estradiol^{88,89} and diltiazem HCl, a drug which has not been previously studied in the literature due to its large molecular weight (415 Da) and ionic, hydrophilic nature.
⁸⁶Johnson, Mark E.; Mitragotri, Samir; Patel, Ashish; Blankschtein, Daniel; Langer, Robert. J. Pharm. Sci. 1996, 85, 670-679

⁸⁷Johnson, Mark E., Blankschtein, Daniel; Langer, Robert. J. Pharm. Sci. 1995, 84, 1144-1146

⁸⁸Chien, Yie W.; Chien, Te-yen; Bagdon, Robert E.; Huang, Yih C.; Bierman, Robert H. Pharm. Res. 1989, 12, 1000-1010

⁸⁹Powers, Marilou S.; Schenkel, Lotte; Darley, Paul E.; Good, William R.; Balestra, Joanne C.; Place, Virgil A. Am. J. Obstet. Gynecol. 1985, 152, 1099-1106

B. Materials

Drugs: Lidocaine free base, Lidocaine HCl, Estradiol, and Diltiazem HCl were purchased from Sigma (St. Louis, Mo.). Chemicals: NMP was a generous gift from ISP Technologies, Inc. (Wayne, N.J.). Polysorbate 80 NF, HLB=15.0 (Tween 80) was purchased from Advance Scientific & Chem. (Ft. Lauderdale, Fla.). Isopropyl myristate (IPM), oleyl alcohol (99%), anhydrous ethyl alcohol, sorbitan mono-oleate (Span 20), HLB=8.6, and phosphate buffered saline tablets (PBS) were purchased from Sigma (St. Louis, Mo.). HPLC grade solvents were used as received. Skin: Human cadaver skin from the chest, back, and abdominal regions was obtained from the National Disease Research Institute (Philadelphia, Pa.). The skin was stored at −80° C. until use.

C. Methods

(i) Microemulsion Phase Diagrams. Four microemulsion (ME) systems were investigated to determine their ternary phase diagrams. All percentages are given as mass ratios.

| | | Organic | |
|---|---|---|---|
| System | Aqueous Phase | Phase | Surfactant Phase |
| 1 | H₂O:Ethanol (1:1) | IPM | Tween 80 |
| 2 | H₂O:Ethanol (1:1) | IPM | Tween 80:Span 20 (49:51) |
| 3 | H₂O | IPM | Tween 80:Ethanol (1:1) |
| 4 | H₂O | IPM | Tween 80:Ethanol (2:1) |

Each of the three components for a system was titrated until a phase change between microemulsion and two phase mixture was observed. The boundary of this transition was recorded over the entire concentration range. A microemulsion was determined as a miscible, optically clear, stable solution. At the transition to a two phase regime, there is a clear clouding of the mixture as well as an eventual separation of the phases. All microemulsion systems were stable for over 6 months.

(ii) Preparation of Formulations. Sample solutions were prepared in 20 ml glass vials and saturated with drug. Drug flux was tested through ME system 1 at two selected concentrations, one in the W/O region, and the other in the O/W. The W/O system consisted of H₂O:IPM:Tween 80 (10:52:38 w/w) while the O/W system contained H₂O:Ethanol:IPM:Tween 80 (27:18:16:39 w/w). Both systems stably incorporated 10% w/w NMP and 10% w/w oleyl alcohol. Drug concentration in the formulation was generally 4% for lidocaine, 2% for diltiazem HCl, and 0.4% for estradiol. The “water phase” sample consisted of the aqueous elements H₂O:Ethanol:NMP (51:31:18) in the same relative proportions as if the organic components were removed. All vehicles studied formed miscible, single phase liquids.

(iii) Lidocaine Partitioning. The logarithm of the relative partition coefficient between IPM and water (log[IPM/H₂O]) was determined for NMP concentrations of 0-35% (v/v). In a micro-centrifuge tube, 500 μl of IPM was added to 500 μl of ddH₂O with the addition of the appropriate amount of NMP. Lidocaine free base was included at 1.0 mg/ml in the organic (IPM) phase. For lidocaine HCl samples, the drug was dissolved in the aqueous phase at 1.0 mg/ml. The two phase system was thoroughly vortexed and allowed to equilibrate. The samples were then centrifuged at 14,000 rpm for 6 minutes to separate the phases. The concentration of lidocaine in each phase was determined by HPLC.

(iv) Preparation of Skin Samples. Human cadaver skin was thawed at room temperature. The epidermis-SC was separated from the full thickness tissue after immersion in 60° C. water for 2 minutes. Heat stripped skin was immediately mounted on diffusion cells.

(v) Skin Transport Experiments. The skin was mounted onto a side-by-side glass diffusion cell with an inner diameter of 5 mm. The two halves of the cell were clamped shut and both reservoirs were filled with 2 ml of phosphate buffered saline (PBS, 0.01 M phosphate, 0.137 M NaCl, pH 7.4). The integrity of the skin was verified by measuring the electrical conductance across the skin barrier at 1 kHz and 10 Hz at 143.0 mV (HP 33120A Waveform Generator). Skin samples measuring 4-14 μA at 1 kHz were used for the diffusion studies. Prior to introducing the donor solution, the skin sample was thoroughly rinsed with PBS to remove surface contaminants. At t=0, the receiver compartment was filled with 2.0 ml of PBS, while 2.0 ml of sample was added to the donor compartment. Both compartments were continuously stirred to maintain even concentrations. At regular time intervals, 1.0 ml of the receiver compartment was transferred to a glass HPLC vial. The remaining solution in the receiver compartment was thoroughly aspirated and discarded. Fresh PBS (2.0 ml) was dispensed into the receiver compartment to maintain sink conditions. At 21 hours, the experiment was terminated. After both compartments were refilled with PBS, the conductance across the skin membrane was again checked to ensure that the skin was not damaged during the experiment. All flux experiments were conducted in triplicate at room temperature. The observed variability of the individual drug transport values was consistent with the previously established 40% intersubject variability of human skin.⁹⁰
⁹⁰Williams, A. C.; Cornwell, P. A.; Barry, B. W. Int. J. Pharm. 1992, 86, 69-77

(vi) Drug Quantification. Lidocaine was assayed by high pressure liquid chromatography (Shimadzu model HPLC, SCL-10A Controller, LC-10AD pumps, SPD-M10A Diode Array Detector, SIL-10AP Injector, Class VP v.5.032 Integration Software) on a reverse phase column (Waters μBondapak™ C₁₈ 3.9×150 mm) using ddH₂O (5% acetic acid, pH 4.2)/acetonitrile (35:65 v/v) as the mobile phase, under isocratic conditions (1.6 mL/min) by detection at 237 nm. The retention time of lidocaine under these conditions was between 3.4 and 4.3 minutes. Standard solutions were used to generate calibration curves. Diltiazem HCl was quantified on a Waters Symmetry® C₁₈ 5 μm, 3.9×150 mm column (WAT046980). The mobile phase consisted of aqueous phase:acetonitrile:methanol (50:25:25) where the aqueous phase consisted of 1.16 g/L d-10-camphorsulfonic acid, 0.1 M sodium acetate, pH 6.2. The system ran at a flow rate of 1.6 ml/min. Chromatograms were integrated at a peak of 240 nm. Estradiol was quantified on a Waters 4.6×250 mm C₁₈ column. The mobile phase consisted of acetonitrile:water (55:45) at a flow rate of 2.0 m/min. Chromatograms were integrated at a peak of 280 nm.

(vii) Calculations. The total mass of drug transported across the skin was determined by HPLC. The flux equation gives:

$$J = \frac{1}{A} ( \frac{ⅆ M}{ⅆ t} ) = P Δ C$$

where J is flux (μg cm⁻² hr⁻¹), A is cross sectional area of the skin membrane (cm²), P is the apparent permeability coefficient (cm hr⁻¹), and ΔC is the concentration gradient. In this experiment, ΔC is taken as the saturation concentration (given infinite dose and sink conditions), and dM/dt is averaged as the total mass transport over the steady state portion of the transport curve. Statistical analyses were performed by the Student's t-test.
D. Results and Discussion

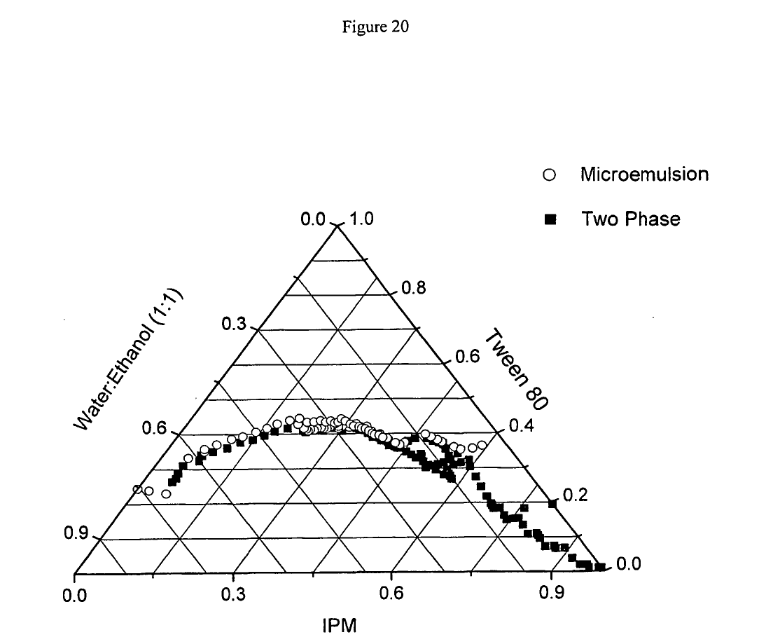

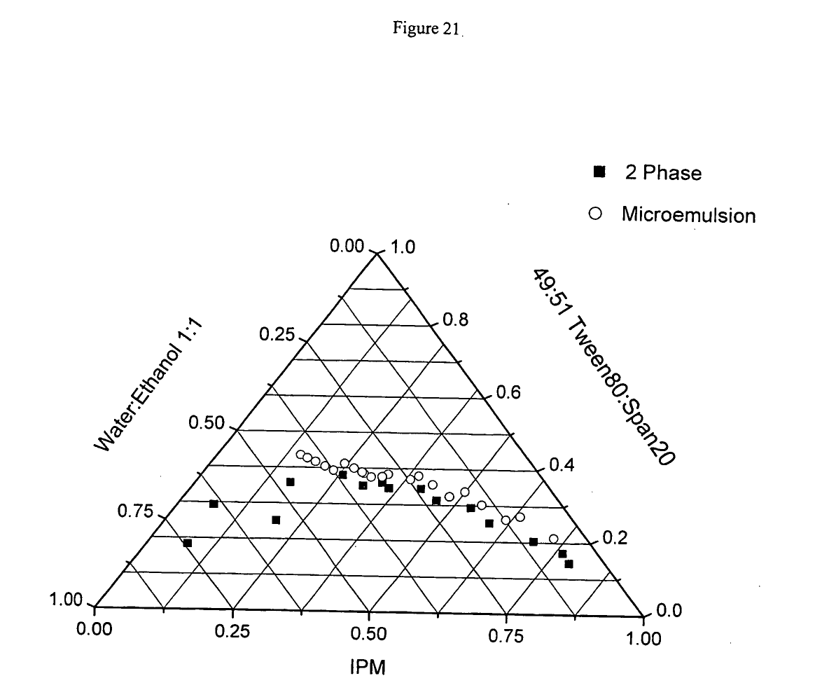

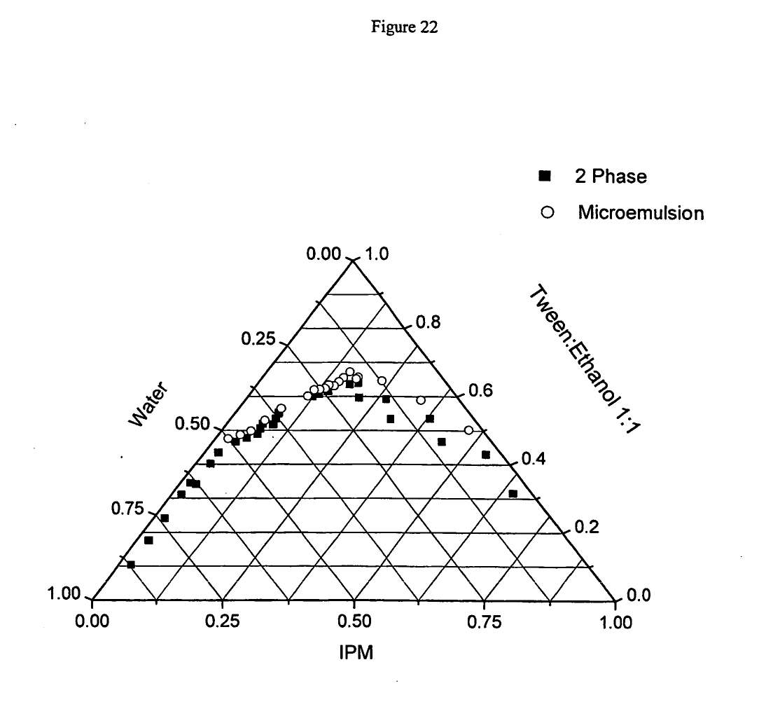

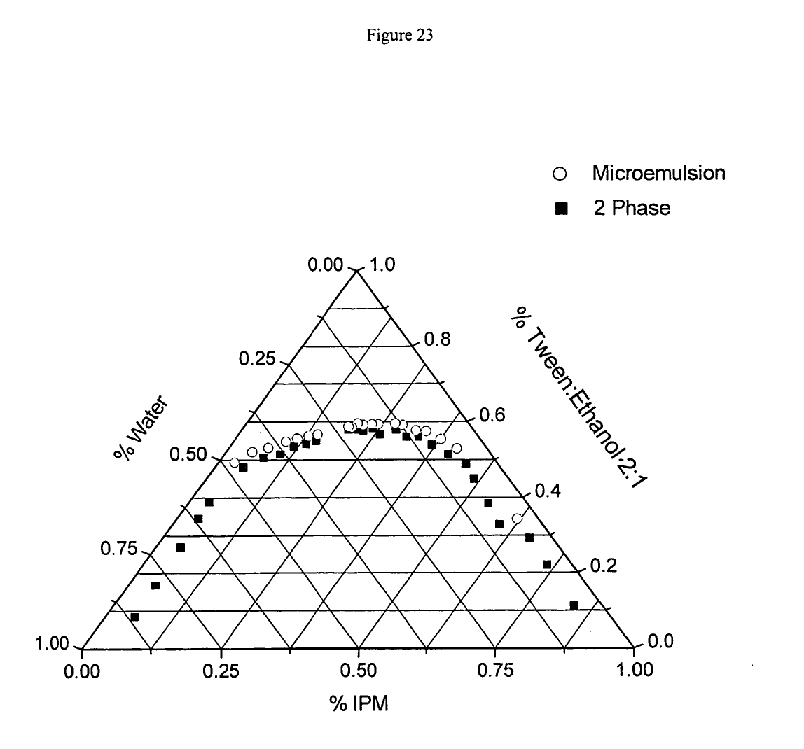

(i) Microemulsion systems. Thermodynamically stable, optically transparent, single phase, liquid formulations were created with the four systems (FIGS. 20-23). An ethanol co-surfactant is necessary to maintain stable O/W emulsions. This is consistent with previous work with ME systems where co-surfactants (usually short chain alcohols) are necessary to maintain a single phase.⁹¹ In system 2, a combination of two nonionic surfactants was used. The mixture of 49:51 w/w Tween 80 (HLB=15) and Span 20 (HLB=8.6) has been reported to act in synergy to maximize water uptake.⁹² Although this system was not tested for transdermal transport, the phase diagram does indeed indicate that ME formation occurs at lower surfactant concentrations. The phase diagrams in FIGS. 22-23 contain the same components as FIG. 20. In these two diagrams, the surfactant/cosurfactant (Tween 80/ethanol) ratio is fixed over the entire range. It is apparent that having too much ethanol is detrimental to ME formation (Table 11). The maximum IPM uptake in O/W ME systems occurs at Tween 80/ethanol ratio of 1:1. Furthermore, it was observed that the cosurfactant was necessary primarily to stabilize ME formulations with high water content. Systems with too little ethanol were unable to form stable O/W microemulsions.
⁹¹Ho, Hsiu-O; Hsiao, Chih-Chuan; Sheu, Ming-Thau. J. Pharm. Sci. 1996, 85(2) 138-143

⁹²Huibers, Paul D. T. Surfactant Self-Assembly, Kinetics and Thermodynamics of Micellar and Microemulsion Systems. Ph.D. Thesis, University of Florida, 1996

All systems could stably incorporate 10% w/w of the transdermal enhancers NMP, oleyl alcohol, oleic acid, or decanoic acid. Drug solubility reached ˜30% w/w lidocaine free base in the W/O system and ˜25% lidocaine HCl in the O/W system. With such high tolerance for the addition of both hydrophilic and hydrophobic molecules, the ME systems studied are robust vehicles for transdermal drug delivery.

(ii) Transdermal Transport. A W/O and O/W formulation from system 1 was selected to test transdermal delivery of hydrophilic and hydrophobic drugs across stripped human skin. The results (Table 12) indicate that the O/W system provided significantly better flux for all the drugs studied (p<0.025). The presence of a second drug in the same ME (estradiol with diltiazem HCl) did not affect the transport of either drug (p>0.5). The permeability of drug from the water phase solution is statistically comparable to that of the O/W ME formulation (p>0.25).

For all the drugs tested, the ME systems provided significant enhancement (Table 14). The finding that flux is improved in O/W formulations as compared with W/O systems even for the hydrophobic drugs suggests that transport from the aqueous phase is key. When the organic phase and surfactants were removed from the ME, leaving only the water phase components (H₂O, ethanol, NMP), the flux was comparable to that from the O/W ME (Table 13). Previous work indicates that the H₂O/NMP synergy provides greater transdermal flux enhancement than H₂O/ethanol.¹⁷ Although the complexity of the multiple components in the system makes it difficult to determine the exact molecular interactions, it appears that the presence of NMP in the water phase plays a key role in the transport of hydrophobic drugs from an O/W ME.
¹⁷Ranade, Vasant V. J. Clin. Pharmacol. 1991, 31, 401-418

It has been previously suggested that ME transdermal enhancement is a result of increasing drug concentration in the donor phase. In our systems containing the chemical enhancer NMP, we believe that the effective permeability of the membrane is also affected. If enhancement is merely a concentration effect, then the permeability of drug across human skin should remain constant. The permeability of all four drugs was compared from the ME systems against the solvent (IPM or H₂O) in which they were most soluble (Table 14). There is a clear permeability enhancement for both hydrophilic and hydrophobic drugs from the ME systems (p<0.001). This finding agrees with previous work where we found that NMP is capable of improving permeability of drugs from both IPM and H₂O.^{16,17}
¹⁶Sleight, P. J. Cardiovasc. Pharmacol. 1990, 16, Suppl 5: S113-119

¹⁷Ranade, Vasant V. J. Clin. Pharmacol. 1991, 31, 401-418

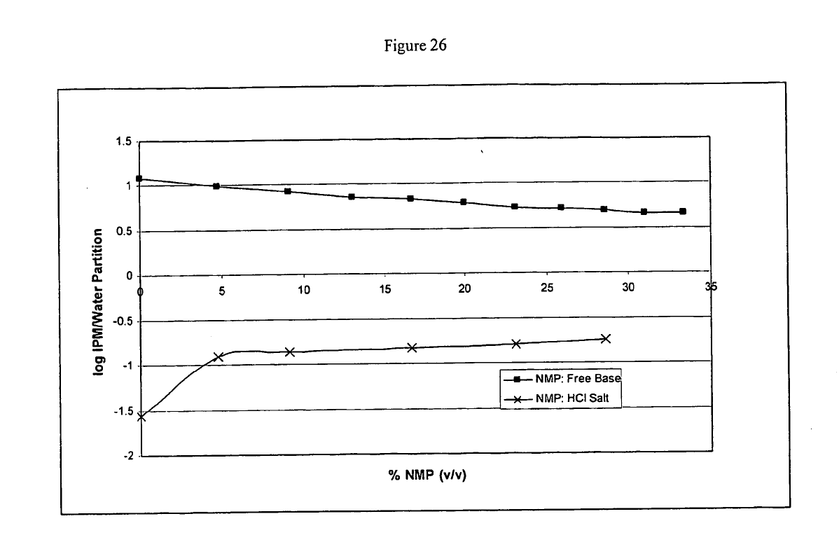

(iii) Effect of NMP on Lidocaine Partitioning. NMP is freely miscible in both H₂O and IPM. It is also capable of improving lidocaine partitioning into the phase where the drug is less soluble (FIG. 26). The hydrophobic lidocaine free base partitions 2.6 times more in the aqueous phase with the addition of 33% NMP. Similarly, the hydrophilic lidocaine HCl partitions 6.5 times more favorably in the IPM phase with the addition of 33% NMP. The concentrations of drug in the minority phase is improved 1.9-fold for lidocaine free base and 5.7-fold for lidocaine HCl. From these results, we can conclude that NMP can act as a partition enhancer in ME systems. In our model for hydrophobic drug transport from an O/W ME, the drug (e.g. lidocaine free base) must first partition from the organic phase into the aqueous phase to reach the skin. The presence of NMP in the system is able to increase the concentration of the hydrophobic drug in the water phase, making it available for transport. Data from FIG. 26 indicates that NMP is also capable of improving the partitioning of hydrophilic drugs to the IPM phase in a W/O ME.

(iv) O/W ME Systems. We propose the following mode of enhancement by NMP in the O/W system. A hydrophobic drug will preferentially partition in the encapsulated organic phase, making flux difficult. The presence of NMP improves partition (and concentration) in the bulk aqueous phase. While in this phase, the drug can favorably partition into the skin with the aid of NMP.¹⁷ For hydrophilic drugs, the presence of NMP in the aqueous phase improves the permeability of the drug across human skin via H₂O/NMP synergy.¹⁷ The role of the organic phase for hydrophilic drug transport from an O/W ME is unknown.
¹⁷Ranade, Vasant V. J. Clin. Pharmacol. 1991, 31, 401-418

We further hypothesize that NMP is a more effective enhancer from the aqueous phase of a ME than the organic phase. NMP was found to have an IPM/H₂O partition ratio of 0.02. Because NMP resides almost exclusively in the water phase of the system, its enhancing effects from that phase should dominate. In a W/O ME, the NMP is sequestered in the encapsulated phase and unable to interact with the skin. This might explain why both the hydrophilic and hydrophobic drugs transport better from the O/W ME. A second mode of hydrophobic drug flux enhancement by NMP from the water phase is also possible. Hydrophobic molecules will not readily leave an organic phase in which they are highly soluble. For this reason, the partition of lidocaine free base from IPM into the skin is slow. However, when lidocaine is in the aqueous phase, it has two partitioning options. It can return to the organic phase, or follow NMP (to which it has high affinity) across the skin membrane. By this account, the water phase of an O/W ME provides a favorable environment for a hydrophobic drug to partition into the skin.

The transdermal delivery of diltiazem HCl has not previously been reported. A drug such as diltiazem HCl is normally precluded from transdermal delivery. Its large molecular weight greatly diminishes its permeability across the skin.⁹³ Ionic drugs have also been proven to be difficult to deliver transdermally.^{94,95,96} Transport of diltiazem HCl from the O/W ME system showed the most drastic enhancement of the 4 drugs tested. This result is promising for delivery of other ionic salt drugs from the ME system.
⁹³Mitragotri, Samir; Johnson, Mark E.; Blankschtein, Daniel; Langer, Robert. Biophysical Journal. 1999, 77, 1268-1283.

⁹⁴Gorukanti, Sudhir R.; Li, Lianli; Kim, Kwon H. Int. J. Pharm. 1999, 192, 159-172

⁹⁵Roy, Samir D.; Roos, Eric; Sharma, Kuldeepak. J. Pharm. Sci. 1994, 83, 126-130

⁹⁶Peck, Kendall D.; Ghanem, Abdel-Halim; Higuchi, William I. J. Pharm. Sci. 1995, 84, 975982

The systems studied provide many interesting characteristics for a transdermal delivery vehicle. They are robust, and stable to the addition of significant amounts of soluble enhancers or excipients. They are capable of enhancing both hydrophilic and hydrophobic drugs, as well as simultaneous delivery of two drugs without diminished flux. The ME systems are also thermodynamically stable, and transport of lidocaine free base after 6 months storage at room temperature was equivalent to its initial value. We believe the novel systems proposed in this study offer a viable vehicle for transdermal drug delivery.

E. Tables

TABLE 11
Maximum IPM Uptake in O/W ME Systems

| 2. Tween 80/Ethanol | % IPM | % Tween |
|---|---|---|
| Ratio | Uptake (w/w) | 80/Ethanol (w/w) |
| 1:2 | 8.3 | 66 |
| 2:3 | 8.1 | 65 |
| 1:1 | 47 | 50 |
| 2:1 | 42 | 53 |
| 4:1 | 4.5 | 56 |
| 9:1 | 1.6 | 58 |

TABLE 12
Lidocaine Free Base and Lidocaine HCl Transport from ME Systems

| | Lidocaine Free Base | | Lidocaine HCl | |
|---|---|---|---|---|
| | Fluxₛₛ | Permeability | Fluxₛₛ | Permeability |
| 3. Formulation | (μg/cm²/hr) | (cm/hr · 10⁵) | (μg/cm²/hr) | (cm/hr · 10⁵) |
| Water | 6.0 ± 1.0 | 133 ± 23 | 0.61 ± 0.38 | 0.61 ± 0.38 |
| W/O ME | 16.5 ± 1.8 | 40.2 ± 4.5 | 2.1 ± 0.2 | 3.5 ± 0.3 |
| O/W ME | 23.3 ± 1.3 | 75.8 ± 4.1 | 10.2 ± 3.9 | 18.1 ± 6.9 |

N = 3

TABLE 13
Estradiol and Diltiazem HCl Transport from ME Systems

| | Estradiol | | Diltiazem HCl | |
|---|---|---|---|---|
| For- | Fluxₛₛ | Permeability | Fluxₛₛ | Permeability |
| mulation | (μg/cm²/hr) | (cm/hr · 10⁵) | (μg/cm²/hr) | (cm/hr · 10⁵) |
| H₂O | 0.015 ± 0.006 | 460 ± 183 | 0.05 ± 0.01 | 0.015 ± 0.004 |
| W/O ME | 0.053 ± 0.029 | 1.1 ± 0.6 | 0.25 ± 0.13 | 1.2 ± 0.6 |
| W/O ME | 0.12 ± 0.06 | 2.4 ± 1.2 | 0.24 ± 0.08 | 1.2 ± 0.4 |
| Both | | | | |
| Drugs | | | | |
| O/W ME | 0.27 ± 0.07 | 5.8 ± 1.5 | 1.6 ± 0.3 | 7.8 ± 1.3 |
| O/W ME | 0.23 ± 0.05 | 5.0 ± 1.2 | 1.6 ± 0.4 | 7.8 ± 1.9 |
| Both | | | | |
| Drugs | | | | |
| Water | | 6.5 ± 1.7 | | 6.1 ± 3.7 |
| Phase | | | | |

N = 3

TABLE 14
Permeability Enhancement of ME Systems

| | Permeability | |
|---|---|---|
| | (cm/hr · 10⁵) | Enhancement |
| Estradiol | | |
| IPM | <0.1 | |
| W/O ME | 1.1 ± 0.6 | >11 |
| O/W ME | 5.8 ± 1.5 | >58 |
| Diltiazem HCl | | |
| H₂O | 0.015 ± 0.004 | |
| W/O ME | 1.2 ± 0.6 | 80 |
| O/W ME | 7.8 ± 1.3 | 520 |
| Lidocaine Free Base | | |
| IPM | 7.2 ± 1.1 | |
| W/O ME | 40.1 ± 4.5 | 5.6 |
| O/W ME | 123 ± 36 | 17 |
| Lidocaine HCl | | |
| H₂O | 0.61 ± 0.38 | |
| W/O ME | 3.5 ± 0.3 | 5.7 |
| O/W ME | 18.1 ± 6.9 | 30 |

N = 3

F. References for Example 4
69. Lawrence, M. J., et. al. Int. Journal of Pharmaceutics. 1998, 111, 63-72
70. Trotta, M.; Gasco, M. R.; Morel, S. Journal of Controlled Release. 1989, 10, 237-243
71. Trotta, M.; Pattarino, F.; Gasco, M. R. Pharmaceutica Acta Helvetiae. 1996, 71, 135-140
72. Osborne, D. W.; Ward, A. J. I.; O'Neill, K. J. J. Pharm. Pharmacol. 1991, 43, 451-454
73. Ho, Hsiu-O; Hsiao, Chih-Chuan; Sheu, Ming-Thau. J. Pharm. Sci. 1996, 85(2) 138-143
74. Guo, Jianxin; Ping, Qineng; Sun, Guoqin; Jiao, Chunhong. Int. J. Pharm. 2000, 194, 201-207
75. Zhang, Qiang; Yie, Guoqing; Li, Yie; Yang, Qingsong; Nagai, T. Int. J. Pharm. 2000, 200, 153-159
76. Baca-Estrada, Maria E.; Foldvari, Marianna; Ewen, Catherine; Badea, Ildiko; Babiuk, Lorne A. Vaccine. 2000, 18, 1847-1854
77. Walters, K. A; Dugard, P. H., Florence, A. T. J. Pharm. Pharmacol. 1981, 33, 207-213
78. Sarpotdar, Pramod P.; Zatz, Joel L. J. Pharm. Sci. 1986, 75, 176-181
79. Francoeur, Michael L.; Golden, Guia M.; Potts, Russell O. Pharm. Res. 1990, 7, 621-627
80. Kim, Dae-Duk; Chien, Yie W. J. Pharm. Sci. 1996, 85, 214-219
81. Sasaki, Hitoshi; Kojima, Masaki; Nakamura, Junzo; Shibasaki, Juichiro. J. Pharm. Pharmacol. 1990, 42, 196-199
82. Phillips, Christine A.; Michniak, Bozena B. J. Pharm. Sci. 1995, 84, 1427-1433
83. Yoneto, Kunio; Li, S. Kevin; Ghanem, Abdel-Halim; Crommelin, Dann J. A.; Higuchi, William I. J. Pharm. Sci. 1995, 84(7), 853-61
84. Lee, Philip J.; Ahmad, Naina; Mitragotri, Samir; Langer, Robert, Shastri, V. Prasad. “Evaluation of Chemical Enhancers in the Transdermal Delivery of Lidocaine.” Submitted Pharm. Res.
85. Lee, Philip J.; Mitragotri, Samir; Langer, Robert, Shastri, V. Prasad. “Chaperoning of Lipid Disrupting Agents and Aqueous Phase Transdermal Enhancement by n-Methyl Pyrrolidone.” Submitted Pharm. Res.
86. Johnson, Mark E.; Mitragotri, Samir; Patel, Ashish; Blankschtein, Daniel; Langer, Robert. J. Pharm. Sci. 1996, 85, 670-679
87. Johnson, Mark E., Blankschtein, Daniel; Langer, Robert. J. Pharm. Sci. 1995, 84, 1144-1146
88. Chien, Yie W.; Chien, Te-yen; Bagdon, Robert E.; Huang, Yih C.; Bierman, Robert H. Pharm. Res. 1989, 12, 1000-1010
89. Powers, Marilou S.; Schenkel, Lotte; Darley, Paul E.; Good, William R.;

Balestra, Joanne C.; Place, Virgil A. Am. J. Obstet. Gynecol. 1985, 152, 1099-1106
90. Williams, A. C.; Cornwell, P. A.; Barry, B. W. Int. J. Pharm. 1992, 86, 69-77
91. Ho, Hsiu-O; Hsiao, Chih-Chuan; Sheu, Ming-Thau. J. Pharm. Sci. 1996, 85(2) 138-143
92. Huibers, Paul D. T. Surfactant Self-Assembly, Kinetics and Thermodynamics of Micellar and Microemulsion Systems. Ph.D. Thesis, University of Florida, 1996
93. Mitragotri, Samir; Johnson, Mark E.; Blankschtein, Daniel; Langer, Robert. Biophysical Journal. 1999, 77, 1268-1283.
94. Gorukanti, Sudhir R.; Li, Lianli; Kim, Kwon H. Int. J. Pharm. 1999, 192, 159-172
95. Roy, Samir D.; Roos, Eric; Sharma, Kuldeepak. J. Pharm. Sci. 1994, 83, 126-130
96. Peck, Kendall D.; Ghanem, Abdel-Halim; Higuchi, William I. J. Pharm. Sci. 1995, 84, 975982

1. A transdermal delivery system comprising a drug formulated with a transport chaperone moiety that reversibly associates with the drug, said chaperone moiety being associated with the drug in the formulation so as to enhance transport of the drug across dermal tissue and releasing the drug after crossing said dermal tissue.

2. The system of claim 1, wherein the transport chaperone is n-methyl pyrrolidone (NMP), octadecene, isopropyl myristate (IPM), oleyl alcohol, oleic acid or a derivative thereof.

3. The system of claim 1, wherein the drug is a lidocaine, a prilocaine, an estradiol or a diltiazem.

4. The system of claim 3, wherein the drug is a free base.

5. The system of claim 1, wherein the drug is lidocaine HCl, lidocaine free base, prilocaine HCl, estradiol or diltiazem HCl.

6. The system of claim 1, wherein the chaperone moiety and drug are associated by ionic, hydrophobic, hydrogen-bonding and/or electrostatic interactions.

7. A microemulsion system for transdermal delivery of a drug, which system solubilizes both hydrophilic and hydrophobic components.

8. The microemulsion system of claim 7, being a cosolvent system including a lipophilic solvent and an organic solvent.

9. The microemulsion system of claim 8, wherein the cosolvents are NMP and IPM.

10. The microemulsion system of claim 7, having an aqueous phase of water and ethanol, an organic phase of isopropyl mystate (IPM) and a surfactant phase of Tween 80.

11. The microemulsion system of claim 10, wherein the aqueous phase is water and ethanol, the organic phase is IPM, and the surfactant phase is Tween 80 and Span 20.

12. The microemulsion system of claim 7, having an aqueous phase, a hydrophobic organic phase, and a surfactant phase.

13. The microemulsion system of claim 7, wherein the system is a water-in-oil system.

14. The microemulsion system of claim 7, wherein the system is an oil-in-water system.
